(19) United States
(12) Patent Application Publication
(10) Pub. No.: US 2009/0253662 A1
(43) Pub. Date: Oct. 8, 2009

(54) GENETIC RISK ASSESSMENT IN HEART FAILURE: IMPACT OF GENETIC VARIATION OF ALDOSTERONE SYNTHASE PROMOTER POLYMORPHISM

(75) Inventors: Manuel Worcel, Boston, MA (US); Michael Sabolinski, Franklin, MA (US); Sang W. Tam, Dover, MA (US); Dennis M. McNamara, Venetia, PA (US)

Correspondence Address: WILMERHALE/NITROMED, 1875 PENNSYLVANIA AVE, NW, WASHINGTON, DC, 20006, US

(73) Assignees: NitroMed, Inc., Lexington, MA (US); University of Pittsburgh of the Commonwealth System of Higher Education, Pittsburgh, PA (US)

(21) Appl. No.: 12/088,924

(22) PCT Filed: Oct. 4, 2006
(86) PCT No.: PCT/US06/38956

§ 371 Date: Dec. 8, 2008

Related U.S. Application Data
(60) Provisional application No. 60/722,995, filed on Oct. 4, 2005.
(60) Provisional application No. 60/776,677, filed on Feb. 27, 2006.

(51) Int. Cl. A61K 31/56; A61K 31/34; A61K 31/502; A61K 31/40; A61K 31/403; A61K 31/352; A61P 9/00

(52) U.S. Cl. 514/171; 514/470; 514/248; 514/423; 514/412; 514/456

(57) ABSTRACT

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (Ic) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; or (r) treating cardiovascular diseases; in a patient in need thereof, wherein the patient has a −344 (T/T) polymorphism or a −344 (C/C) polymorphism in an aldosterone synthase CYP11B2 gene, comprising administering to the patient (i) at least one antioxidant compound or a pharmaceutically acceptable salt thereof; (ii) at least one nitric oxide enhancing compound; and (iii) optionally the best current therapy for the treatment of cardiovascular diseases. In one embodiment the antioxidant is a hydralazine compound or a pharmaceutically acceptable salt thereof and the nitric oxide enhancing compound is isosorbide dinitrate and/or isosorbide mononitrate.

### RELATED APPLICATIONS

This application claims priority under 35 USC § 119 to U.S. Application No. 60/722,995 filed Oct. 4, 2005, and U.S. Application No. 60/776,677 filed Feb. 27, 2006; the disclosures of each of which are incorporated by reference herein in their entirety.

### FIELD OF THE INVENTION

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; or (r) treating cardiovascular diseases; in a patient in need thereof, wherein the patient has a −344 (T/T) polymorphism or a −344 (C/C) polymorphism in an aldosterone synthase CYP11B2 gene, comprising administering to the patient (i) at least one antioxidant compound or a pharmaceutically acceptable salt thereof; (ii) at least one nitric oxide enhancing compound; and (iii) optionally the best current therapy for the treatment of cardiovascular diseases. In one embodiment the antioxidant is a hydralazine compound or a pharmaceutically acceptable salt thereof and the nitric oxide enhancing compound is isosorbide dinitrate and/or isosorbide mononitrate.

### BACKGROUND OF THE INVENTION

Activation of the renin angiotensin system worsens heart failure progression, and increases in circulating aldosterone play a central role. Stimulation of myocardial aldosterone receptors increases apoptosis, resulting in fibrosis and ventricular remodeling. Blockade of the aldosterone has been shown to improve heart failure outcomes. In the RALES trial the addition of the aldosterone receptor antagonist spironolactone improved survival in subjects with severe heart failure. Aldosterone antagonists also reduce left ventricular remodeling and the addition of the selective antagonist eplerenone post myocardial infarction improves survival.

Significant clinical heterogeneity exists in heart failure outcomes and much of this variability is genetically based. Aldosterone synthase (CYP11B2) is a 9 exon gene occurring on chromosome 8q22. A common single nucleotide polymorphism, C to T transition for position −344, occurs within the promoter region of CYP11B2. The −344C allele binds the steroidogenic transcription factor 1 (SF-1) 4 times more than the T allele, and has been linked to increased aldosterone production. The CYP11B2 promoter polymorphism has been linked to hypertension and the −344 C allele in particular to the risk of coronary disease. Despite the central role of aldosterone in heart failure progression, the impact of the −344C allele on clinical outcomes is unknown.

There is a need in the art for the determination of a patient's genetic variation and for the treatment of heart failure.

### SUMMARY OF THE INVENTION

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, comprising administering to the patient (i) at least one antioxidant compound or pharmaceutically acceptable salt thereof, (ii) at least one nitric oxide enhancing compound; and (iii) optionally at least one compound selected from the group consisting of an angiotensin converting enzyme inhibitor, a β-adrenergic antagonist, an angiotensin II antagonist, an aldosterone antagonist, a cardiac glycoside and a diuretic compound or a combination of two or more thereof. In another embodiment the patient has at least one polymorphism in the endothelial nitric oxide synthase (NOS3) gene and/or at least one polymorphism in the beta 1 adrenergic receptor gene. In another embodiment, the patient is categorized as New York Heart Association heart failure functional classification I, II, III or IV. In yet another embodiment, the patient is categorized as New York Heart Association heart failure functional classification II, III or IV. In yet another embodiment the patient is a black patient. In one embodiment the antioxidant is a hydralazine compound or a pharmaceutically acceptable salt thereof and the nitric oxide enhancing compound is isosorbide dinitrate and/or isosorbide mononitrate. The antioxidants, nitric oxide enhancing compounds and/or additional compounds can be administered separately or as components of the same composition in one or more pharmaceutically acceptable carriers.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, comprising administering to the patient (i) at least one antioxidant compound or pharmaceutically acceptable salt thereof; (ii) at least one nitric oxide enhancing compound; (iii) an aldosterone antagonist; and (iv) optionally at least one compound selected from the group consisting of an angiotensin converting enzyme inhibitor, a β-adrenergic antagonist, an angiotensin II antagonist, a cardiac glycoside and a diuretic compound or a combination of two or more thereof. In another embodiment the patient has at least one polymorphism in the endothelial nitric oxide synthase (NOS3) gene and/or at least one polymorphism in the beta 1 adrenergic receptor gene. In one embodiment the antioxidant is a hydralazine compound or a pharmaceutically acceptable salt thereof and the nitric oxide enhancing compound is isosorbide dinitrate and/or isosorbide mononitrate. In these embodiments of the invention, the methods can involve (i) administering the hydralazine compound or a pharmaceutically acceptable salt thereof, and at least one of isosorbide dinitrate and/or isosorbide mononitrate, and an aldosterone antagonist or (ii) administering the hydralazine compound or a pharmaceutically acceptable salt thereof, at least one of isosorbide dinitrate and/or isosorbide mononitrate, an aldosterone antagonist, and at least one compound selected from the group consisting of an angiotensin converting enzyme inhibitor, a β-adrenergic antagonist, an angiotensin II antagonist, a cardiac glycoside and a diuretic compound. or a combination of two or more thereof. In another embodiment the patient has at least one polymorphism in the endothelial nitric oxide synthase (NOS3) gene and/or at least one polymorphism in the beta 1 adrenergic receptor gene. In another embodiment, the patient is categorized as New York Heart Association heart failure functional classification I, II, III or IV; e.g., II, III or IV. In yet another embodiment the patient is a black patient. In one embodiment the antioxidant is a hydralazine compound or a pharmaceutically acceptable salt thereof and the nitric oxide enhancing compound is isosorbide dinitrate and/or isosorbide mononitrate. The antioxidants, nitric oxide enhancing compounds and/or additional compounds can be administered separately or as components of the same composition in one or more pharmaceutically acceptable carriers.

These and other aspects of the invention are described in detail herein.

### BRIEF DESCRIPTION OF THE FIGURES

FIG. 1 shows the genotype frequencies for the aldosterone synthase (CYP11B2) −344 T/C polymorphism in the white heart failure cohort in GRACE and the African American heart failure cohort form GRAHF. The prevalence of the T allele in significantly higher (p<0.001) in African Americans

FIG. 2 shows the event free survival by CYP11B2-344 genotype subsets. Freedom from heart failure hospitalization was significantly poorer (p=0.03) in subjects with the C allele, with the worst outcomes in CC homozygotes, best outcomes in TT subjects and intermediate in heterozygotes.

FIG. 3 shows the effect of the administration of a combination of hydralazine hydrochloride and isosorbide dinitrate on outcomes in heart failure. FIG. 3A shows the effect on the composite score in −344 genotype subsets. Treatment was associated with marked improvement in the −344 TT subset (n=218, p=0.01), but minimal effect in subjects with the −344 C allele (CC+TC, n=136, p=ns). FIG. 3B shows the effect on change in quality of life scores from baseline at 6 months in −344 genotype subsets.

### DETAILED DESCRIPTION OF THE INVENTION

As used throughout the disclosure, the following terms, unless otherwise indicated, shall be understood to have the following meanings.

“Patient” refers to animals, preferably mammals, most preferably humans, and includes males and females.

“Black” refers to a person of African descent or an African-American person. A person may be African-American or black if he/she designates himself/herself as such.

“Effective amount” refers to the amount of the compound and/or composition that is necessary to achieve its intended purpose.

“Heart failure” includes, but is not limited to congestive heart failure, compensated heart failure, decompensated heart failure, and the like.

“Compensated heart failure” refers to a condition in which the heart functions at an altered, but stable physiologic state, e.g. at a different but stable point on the Frank-Starling-curve through an increase in preload or after development of myocardial hypertrophy. Compensated heart failure can result in multiple complications, such as progressive increase in capillary related edema, progressive renal failure, or progressive ischemic tissue damage.

“Decompensated heart failure” refers to a condition in which the heart functions at an altered and unstable physiologic state in which cardiac function and related or dependent physiologic functions deteriorate progressively, slowly or rapidly. Decompensated heart failure can result in multiple complications, such as progressive increase in capillary related edema, progressive renal failure, or progressive ischemic tissue damage.

“Reducing hospitalizations related to heart failure” includes but is not limited to prolonging time to hospitalization for heart failure; prolonging time to first hospitalization for heart failure; reducing the total number of days a patient with heart failure spends in the hospital for heart failure for a single hospital stay (i.e., reducing the duration of a single hospital stay for a patient with heart failure); reducing the total number of days a patient spends in the hospital for heart failure for multiple hospital stays (i.e., two or more hospital stays); reducing the number of hospital admissions for heart failure; and the like.

“Oxygen consumption” can be measured during a progressive maximal bicycle-ergometer exercise test taken while the expired air is collected continuously to monitor oxygen consumption. Dyspnea or fatigue typically occurs at a peak oxygen consumption of <25 ml per kilogram of body weight per minute. Patients with pulmonary diseases, obstructive valvular diseases and the like, tend to have a low oxygen consumption. An increase in a patient's oxygen consumption typically results in the patient's increased exercise tolerance and would imply that the patient would have an improved quality of life.

“Quality of life” refers to one or more of a person's ability to walk, climb stairs, do errands, work around the house, participate in recreational activities, and/or not requiring rest during the day, and/or the absence of sleeping problems or shortness of breath. The quality of life can be measured using the Minnesota Living with Heart Failure questionnaire. The questionnaire is self-administered after brief standardization instructions. The score is obtained by summing the ranks of the responses to each question.

“Cardiovascular disease or disorder” refers to any cardiovascular disease or disorder known in the art, including, but not limited to, heart failure, restenosis, hypertension (e.g. pulmonary hypertension, systolic hypertension, labile hypertension, idiopathic hypertension, low-renin hypertension, salt-sensitive hypertension, low-renin, salt-sensitive hypertension, thromboembolic pulmonary hypertension; pregnancy-induced hypertension; renovascular hypertension; hypertension-dependent end-stage renal disease, hypertension associated with cardiovascular surgical procedures, hypertension with left ventricular hypertrophy, and the like), diastolic dysfunction, coronary artery disease, myocardial infarctions, cerebral infarctions, arterial stiffness, atherosclerosis, atherogenesis, cerebrovascular disease, angina, (including chronic, stable, unstable and variant (Prinzmetal) angina pectoris), aneurysm, ischemic heart disease, cerebral ischemia, myocardial ischemia, thrombosis, platelet aggregation, platelet adhesion, smooth muscle cell proliferation, vascular or non-vascular complications associated with the use of medical devices, wounds associated with the use of medical devices, vascular or non-vascular wall damage, peripheral vascular disease, neointimal hyperplasia following percutaneous transluminal coronary angiograph, vascular grafting, coronary artery bypass surgery, thromboembolic events, post-angioplasty restenosis, coronary plaque inflammation, hypercholesterolemia, embolism, stroke, shock, arrhythmia, atrial fibrillation or atrial flutter, thrombotic occlusion and reclusion cerebrovascular incidents, left ventricular dysfunction and hypertrophy, and the like.

“Diseases resulting from oxidative stress” refers to any disease that involves the generation of free radicals or radical compounds, such as, for example, atherogenesis, atheromatosis, arteriosclerosis, atherosclerosis, vascular hypertrophy associated with hypertension, hyperlipoproteinaemia, normal vascular degeneration through aging, parathyroidal reactive hyperplasia, renal disease (e.g., acute or chronic), neoplastic diseases, inflammatory diseases, neurological and acute bronchopulmonary disease, tumorigenesis, ischemia-reperfusion syndrome, arthritis, sepsis, cognitive dysfunction, endotoxic shock, endotoxin-induced organ failure, and the like.

“Endothelial dysfunction” refers to the impaired ability in any physiological processes carried out by the endothelium, in particular, production of nitric oxide regardless of cause. It may be evaluated by, such as, for example, invasive techniques, such as, for example, coronary artery reactivity to acetylcholine or methacholine, and the like, or by noninvasive techniques, such as, for example, blood flow measurements, brachial artery flow dilation using cuff occlusion of the arm above or below the elbow, brachial artery ultrasonography, imaging techniques, measurement of circulating biomarkers, such as, asymmetric dimethylarginine (ADMA), and the like. For the latter measurement the endothelial-dependent flow-mediated dilation will be lower in patients diagnosed with an endothelial dysfunction.

“Methods for treating endothelial dysfunction” include, but are not limited to, treatment prior to the onset/diagnosis of a disease that is caused by or could result from endothelial dysfunction, such as, for example, atherosclerosis, hypertension, diabetes, heart failure, and the like.

“Methods for treating diseases caused by endothelial dysfunction” include, but are not limited to, the treatment of any disease resulting from the dysfunction of the endothelium, such as, for example, arteriosclerosis, heart failure, hypertension, cardiovascular diseases, cerebrovascular diseases, renovascular diseases, mesenteric vascular diseases, pulmonary vascular diseases, ocular vascular diseases, peripheral vascular diseases, peripheral ischemic diseases, and the like.

“Renovascular diseases” refers to any disease or dysfunction of the renal system including, but not limited to, renal failure (e.g., acute or chronic), renal insufficiency, nephrotic edema, acute glomerulonephritis, oliguric renal failure, renal deterioration associated with severe hypertension, unilateral perechymal renal disease, polycystic kidney disease, chronic pyelonephritis, renal diseases associated with renal insufficiency, complications associated with dialysis or renal transplantation, renovascular hypertension, nephropathy, glomerulonephritis, scleroderma, glomerular sclerosis, renal artery stenosis, AIDS-associated nephropathy, immune-mediated renal disease, atheroembolic renal disease, pre-renal azotemia, and the like.

“Prodrug” refers to a compound that is made more active in vivo.

“Angiotensin converting enzyme (ACE) inhibitor” refers to compounds that inhibit an enzyme which catalyzes the conversion of angiotensin I to angiotensin II. ACE inhibitors include, but are not limited to, amino acids and derivatives thereof, peptides, including di- and tri-peptides, and antibodies to ACE which intervene in the renin-angiotensin system by inhibiting the activity of ACE thereby reducing or eliminating the formation of the pressor substance angiotensin II.

“Angiotensin II antagonists” refers to compounds which interfere with the function, synthesis or catabolism of angiotensin II. Angiotensin II antagonists include peptide compounds and non-peptide compounds, including, but not limited to, angiotensin II antagonists, angiotensin II receptor antagonists, agents that activate the catabolism of angiotensin II, and agents that prevent the synthesis of angiotensin I from angiotensin II. The renin-angiotensin system is involved in the regulation of hemodynamics and water and electrolyte balance. Factors that lower blood volume, renal perfusion pressure, or the concentration of sodium in plasma tend to activate the system, while factors that increase these parameters tend to suppress its function.

“Diuretic compound” refers to and includes any compound or agent that increases the amount of urine excreted by a patient.

“Carriers” or “vehicles” refers to carrier materials suitable for compound administration and include any such material known in the art such as, for example, any liquid, gel, solvent, liquid diluent, solubilizer, or the like, which is non-toxic and which does not interact with any components of the composition in a deleterious manner.

“Sustained release” refers to the release of an active compound and/or composition such that the blood levels of the active compound are maintained within a desirable range over a period of time. The sustained release formulation can be prepared using any conventional method known to one skilled in the art to obtain the desired release characteristics.

“Nitric oxide enhancing” refers to compounds and functional groups which, under physiological conditions can increase endogenous nitric oxide. Nitric oxide enhancing compounds include, but are not limited to, nitric oxide releasing compounds, nitric oxide donating compounds, nitric oxide donors, nitric oxide adducts, radical scavenging compounds and/or reactive oxygen species scavenger compounds. In one embodiment the radical scavenging compound contains a nitroxide group.

“Nitroxide group” refers to compounds that have the ability to mimic superoxide dimutase and catalase and act as radical scavengers, or react with superoxide or other reactive oxygen species via a stable aminoxyl radical i.e. N-oxide.

“Nitric oxide adduct” or “NO adduct” refers to compounds and functional groups which, under physiological conditions, can donate, release and/or directly or indirectly transfer any of the three redox forms of nitrogen monoxide (NO⁺, NO⁻, NO.), such that the biological activity of the nitrogen monoxide species is expressed at the intended site of action.

“Nitric oxide releasing” or “nitric oxide donating” refers to methods of donating, releasing and/or directly or indirectly transferring any of the three redox forms of nitrogen monoxide (NO⁺, NO⁻, NO.), such that the biological activity of the nitrogen monoxide species is expressed at the intended site of action.

“Nitric oxide donor” or “NO donor” refers to compounds that donate, release and/or directly or indirectly transfer a nitrogen monoxide species, and/or stimulate the endogenous production of nitric oxide or endotheliam-derived relaxing factor (EDRF) in vivo and/or elevate endogenous levels of nitric oxide or EDRF in vivo and/or are oxidized to produce nitric oxide and/or are substrates for nitric oxide synthase and/or cytochrome P450. Nitric oxide donors also include compounds that are precursors of L-arginine, inhibitors of the enzyme arginase and nitric oxide mediators.

“Hydralazine compound” refers to a compound having the formula:

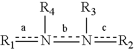

wherein a, b and c are each independently a single or a double bond; R₁ and R₂ are each independently a hydrogen, an alkyl, an ester or a heterocyclic ring; R₃ and R₄ are each independently a lone pair of electrons or a hydrogen, with the proviso that at least one of R₁, R₂, R₃ and R₄ is not a hydrogen. Exemplary hydralazine compounds include budralazine, cadralazine, dihydralazine, endralazine, hydralazine, pildralazine, todralazine and the like.

“Alkyl” refers to a lower alkyl group, a substituted lower alkyl group, a haloalkyl group, a hydroxyalkyl group, an alkenyl group, a substituted alkenyl group, an alkynyl group, a bridged cycloalkyl group, a cycloalkyl group or a heterocyclic ring, as defined herein. An alkyl group may also comprise one or more radical species, such as, for example a cycloalkylalkyl group or a heterocyclicalkyl group.

“Lower alkyl” refers to branched or straight chain acyclic alkyl group comprising one to about ten carbon atoms (preferably one to about eight carbon atoms, more preferably one to about six carbon atoms). Exemplary lower alkyl groups include methyl, ethyl, n-propyl, isopropyl, n-butyl, isobutyl, sec-butyl, t-butyl, pentyl, neopentyl, iso-amyl, hexyl, octyl, and the like.

“Substituted lower alkyl” refers to a lower alkyl group, as defined herein, wherein one or more of the hydrogen atoms have been replaced with one or more R¹⁰⁰ groups, wherein each R¹⁰⁰ is independently a hydroxy, an ester, an amidyl, an oxo, a carboxyl, a carboxamido, a halo, a cyano, a nitrate, a nitrite, a thionitrate, a thionitrite or an amino group, as defined herein.

“Haloalkyl” refers to a lower alkyl group, an alkenyl group, an alkynyl group, a bridged cycloalkyl group, a cycloalkyl group or a heterocyclic ring, as defined herein, to which is appended one or more halogens, as defined herein. Exemplary haloalkyl groups include trifluoromethyl, chloromethyl, 2-bromobutyl, 1-bromo-2-chloro-pentyl, and the like.

“Alkenyl” refers to a branched or straight chain C₂-C₁₀ hydrocarbon (preferably a C₂-C₈ hydrocarbon, more preferably a C₂-C₆ hydrocarbon) that can comprise one or more carbon-carbon double bonds. Exemplary alkenyl groups include propylenyl, buten-1-yl, isobutenyl, penten-1-yl, 2,2-methylbuten-1-yl, 3-methylbuten-1-yl, hexan-1-yl, hepten-1-yl, octen-1-yl, and the like.

“Lower alkenyl” refers to a branched or straight chain C₂-C₄ hydrocarbon that can comprise one or two carbon-carbon double bonds.

“Substituted alkenyl” refers to a branched or straight chain C₂-C₁₀ hydrocarbon (preferably a C₂-C₈ hydrocarbon, more preferably a C₂-C₆ hydrocarbon) which can comprise one or more carbon-carbon double bonds, wherein one or more of the hydrogen atoms have been replaced with one or more R¹⁰⁰ groups, wherein each R¹⁰⁰ is independently a hydroxy, an oxo, a carboxyl, a carboxamido, a halo, a cyano or an amino group, as defined herein.

“Alkynyl” refers to an unsaturated acyclic C₂-C₁₀ hydrocarbon (preferably a C₂-C₈ hydrocarbon, more preferably a C₂-C₆ hydrocarbon) that can comprise one or more carbon-carbon triple bonds. Exemplary alkynyl groups include ethynyl, propynyl, butyn-1-yl, butyn-2-yl, pentyl-1-yl, pentyl-2-yl, 3-methylbutyn-1-yl, hexyl-1-yl, hexyl-2-yl, hexyl-3-yl, 3,3-dimethyl-butyn-1-yl, and the like.

“Bridged cycloalkyl” refers to two or more cycloalkcyl groups, heterocyclic groups, or a combination thereof fused via adjacent or non-adjacent atoms. Bridged cycloalkyl groups can be unsubstituted or substituted with one, two or three substituents independently selected from alkyl, alkoxy, amino, alkylamino, dialkylamino, hydroxy, halo, carboxyl, alkylcarboxylic acid, aryl, amidyl, ester, alkylcarboxylic ester, carboxamido, alkylcarboxamido, oxo and nitro. Exemplary bridged cycloalkyl groups include adamantyl, decahydronapthyl, quinuclidyl, 2,6-dioxabicyclo(3.3.0)octane, 7-oxabicyclo(2.2.1)heptyl, 8-azabicyclo(3,2,1)oct-2-enyl and the like.

“Cycloalkyl” refers to a saturated or unsaturated cyclic hydrocarbon comprising from about 3 to about 10 carbon atoms. Cycloalkyl groups can be unsubstituted or substituted with one, two or three substituents independently selected from alkyl, alkoxy, amino, alkylamino, dialkylamino, arylamino, diarylamino, alkylarylamino, aryl, amidyl, ester, hydroxy, halo, carboxyl, alkylcarboxylic acid, alkylcarboxylic ester, carboxamido, alkylcarboxamido, oxo, alkylsulfinyl, and nitro. Exemplary cycloalkyl groups include cyclopropyl, cyclobutyl, cyclopentyl, cyclohexyl, cyclohexenyl, cyclohepta-1,3-dienyl, and the like.

“Heterocyclic ring or group” refers to a saturated or unsaturated cyclic hydrocarbon group having about 2 to about 10 carbon atoms (preferably about 4 to about 6 carbon atoms) where 1 to about 4 carbon atoms are replaced by one or more nitrogen, oxygen and/or sulfur atoms. Sulfur may be in the thio, sulfinyl or sulfonyl oxidation state. The heterocyclic ring or group can be fused to an aromatic hydrocarbon group. Heterocyclic groups can be unsubstituted or substituted with one, two or three substituents independently selected from alkyl, alkoxy, amino, alkylthio, aryloxy, arylthio, arylalkyl, hydroxy, oxo, thial, halo, carboxyl, carboxylic ester, alkylcarboxylic acid, alkylcarboxylic ester, aryl, arylcarboxylic acid, arylcarboxylic ester, amidyl, ester, alkylcarbonyl, arylcarbonyl, alkylsulfinyl, carboxamido, alkylcarboxamido, arylcarboxamido, sulfonic acid, sulfonic ester, sulfonamide nitrate and nitro. Exemplary heterocyclic groups include pyrrolyl, furyl, thienyl, 3-pyrrolinyl, 4,5,6-trihydro-2H-pyranyl, pyridinyl, 1,4-dihydropyridinyl, pyrazolyl, triazolyl, pyrimidinyl, pyridazinyl, oxazolyl, thiazolyl, imidazolyl, indolyl, thiophenyl, furanyl, tetrahydrofuranyl, tetrazolyl, pyrrolinyl, pyrrolindinyl, oxazolindinyl 1,3-dioxolanyl, imidazolinyl, imidazolindinyl, pyrazolinyl, pyrazolidinyl, isoxazolyl, isothiazolyl, 1,2,3-oxadiazolyl, 1,2,3-triazolyl, 1,3,4-thiadiazolyl, 2H-pyranyl, 4H-pyranyl, piperidinyl, 1,4-dioxanyl, morpholinyl, 1,4-dithianyl, thiomorpholinyl, pyrazinyl, piperazinyl, 1,3,5-triazinyl, 1,3,5-trithianyl, benzo(b)thiophenyl, benzimidazolyl, benzothiazolinyl, quinolinyl, 2,6-dioxabicyclo(3.3.0)octane, and the like.

“Heterocyclic compounds” refer to mono- and polycyclic compounds comprising at least one aryl or heterocyclic ring.

“Aryl” refers to a monocyclic, bicyclic, carbocyclic or heterocyclic ring system comprising one or two aromatic rings. Exemplary aryl groups include phenyl, pyridyl, napthyl, quinoyl, tetrahydronaphthyl, furanyl, indanyl, indenyl, indoyl, and the like. Aryl groups (including bicyclic aryl groups) can be unsubstituted or substituted with one, two or three substituents independently selected from alkyl, alkoxy, alkylthio, amino, alkylamino, dialkylamino, arylamino, diarylamino, alkylarylamino, halo, cyano, alkylsulfinyl, hydroxy, carboxyl, carboxylic ester, alkylcarboxylic acid, alkylcarboxylic ester, aryl, arylcarboxylic acid, arylcarboxylic ester, alkylcarbonyl, arylcarbonyl, amidyl, ester, carboxamido, alkylcarboxamido, carbornyl, sulfonic acid, sulfonic ester, sulfonamido and nitro. Exemplary substituted aryl groups include tetrafluorophenyl, pentafluorophenyl, sulfonamide, alkylsulfonyl, arylsulfonyl, and the like.

“Hydroxy” refers to —OH.

“Hydroxyalkyl” refers to a hydroxy group, as defined herein, appended to an alkyl group, as defined herein.

“Alkylcarbonyl” refers to R₅₂—C(O)—, wherein R₅₂ is an alkyl group, as defined herein.

“Arylcarbonyl” refers to R₅₅—C(O)—, wherein R₅₅ is an aryl group, as defined herein.

“Ester” refers to R₅₁C(O)O— wherein R₅₁ is a hydrogen atom, an alkyl group, an aryl group, an alkylaryl group, or an arylheterocyclic ring, as defined herein.

“Alkylaryl” refers to an alkyl group, as defined herein, to which is appended an aryl group, as defined herein. Exemplary alkylaryl groups include benzyl, phenylethyl, hydroxybenzyl, fluorobenzyl, fluorophenylethyl, and the like.

“Arylheterocyclic ring” refers to a bi- or tricyclic ring comprised of an aryl ring, as defined herein, appended via two adjacent carbon atoms of the aryl ring to a heterocyclic ring, as defined herein. Exemplary arylheterocyclic rings include dihydroindole, 1,2,3,4-tetra-hydroquinoline, and the like.

“Hydrazino” refers to H₂N—N(H)—.

In one embodiment of the invention, the antioxidants include, but are not limited to, small-molecule antioxidants and antioxidant enzymes. Suitable small-molecule antioxidants include, but are not limited to, hydralazine compounds, glutathione, vitamin C, vitamin E, cysteine, N-acetyl-cysteine, β-carotene, ubiquinone, ubiquinol-10, tocopherols, coenzyme Q, superoxide dismutase mimetics, such as, for example, 2,2,6,6-tetramethyl-1-piperidinyloxy (TEMPO), DOXYL, PROXYL nitroxide compounds; 4-hydroxy-2,2,6,6-tetramethyl-1-piperidinyloxy (Tempol), M-40401, M-40403, M-40407, M-40419, M-40484, M-40587, M-40588, and the like. Suitable antioxidant enzymes include, but are not limited to, superoxide dismutase, catalase, glutathione peroxidase, NADPH oxidase inhibitors, such as, for example, apocynin, aminoguanidine, ONO 1714, S17834 (benzo(b)pyran-4-one derivative), and the like; xanthine oxidase inhibitors, such as, for example, allopurinol, oxypurinol, amflutizole, diethyldithiocarbamate, 2-styrylchromones, chrysin, luteolin, kaempferol, quercetin, myricetin, isorhamnetin, benzophenones such as 2,2′,4,4′-tetrahydroxybenzophenone, 3,4,5,2′,3′,4′-hexahydroxybenzophenone and 4,4′-dihydroxybenzophenone; benzothiazinone analogues such as 2-amino-4H-1,3-benzothiazine-4-one, 2-guanidino-4H-1,3-benzothiazin-4-one and rhodanine; N-hydroxyguanidine derivative such as, PR5 (1-(3,4-dimethoxy-2-chlorobenzylideneamino)-3-hydroxyguanidine); 6-formylpterin, and the like. The antioxidant enzymes can be delivered by gene therapy as a viral vector and/or a non-viral vector. Suitable antioxidants are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

In some embodiments the antioxidants are apocynin, hydralazine compounds and superoxide dimutase mimetics. In one embodiment, the hydralazine compound is budralazine, cadralazine, dihydralazine, endralazine, hydralazine, pildralazine, todralazine or a pharmaceutically acceptable salt thereof. In another embodiment the hydralazine compound is hydralazine. In yet another embodiment, the pharmaceutically acceptable salt of hydralazine is hydralazine hydrochloride. Hydralazine hydrochloride is commercially available from, for example, Lederle Standard Products, Pearl River, N.Y.; and Par Pharmaceuticals Inc., Spring Valley, N.Y. It is a white to off-white, crystalline powder and is soluble in water, slightly soluble in alcohol and very slightly soluble in ether.

The antioxidants, such as, hydralazine compounds, are used in combination with nitric oxide enhancing compounds that release nitric oxide, increase endogeneous levels of nitric oxide or otherwise directly or indirectly deliver or transfer a biologically active form of nitrogen monoxide to a site of its intended activity, such as on a cell membrane in vivo.

Nitrogen monoxide can exist in three forms: NO— (nitroxyl), NO. (nitric oxide) and NO⁺ (nitrosonium). NO. is a highly reactive short-lived species that is potentially toxic to cells. This is critical because the pharmacological efficacy of NO depends upon the form in which it is delivered. In contrast to the nitric oxide radical (NO.), nitrosonium (NO⁺) does not react with O₂ or O₂— species, and functionalities capable of transferring and/or releasing NO⁺ and NO— are also resistant to decomposition in the presence of many redox metals. Consequently, administration of charged NO equivalents (positive and/or negative) does not result in the generation of toxic by-products or the elimination of the active NO group.

The term “nitric oxide” encompasses uncharged nitric oxide (NO.) and charged nitrogen monoxide species, such as nitrosonium ion (NO⁺) and nitroxyl ion (NO—). The reactive form of nitric oxide can be provided by gaseous nitric oxide. The nitrogen monoxide releasing, delivering or transferring compounds have the structure F—NO, wherein F is a nitrogen monoxide releasing, delivering or transferring group, and include any and all such compounds which provide nitrogen monoxide to its intended site of action in a form active for its intended purpose.

The term “nitric oxide donor compounds” encompasses any nitrogen monoxide releasing, delivering or transferring compounds, including, for example, S-nitrosothiols, nitrites, nitrates, S-nitrothiols, sydnonimines, 2-hydroxy-2-nitrosohydrazines, (NONOates), (E)-alkyl-2-((E)-hydroxyimino)-5-nitro-3-hexeneamide (FK-409), (E)-alkyl-2-((E)-hydroxyimino)-5-nitro-3-hexeneamines, N-((2Z,3E)-4-ethyl-2-(hydroxyimino)-6-methyl-5-nitro-3-heptenyl)-3-pyridinecarboxamide (FR 146801), N-nitrosoamines, N-hydroxyl nitrosamines, nitrosimines, diazetine dioxides, oxatriazole 5-imines, oximes, hydroxylamines, N-hydroxyguanidines, hydroxyureas, benzofuroxanes, fluoroxans as well as substrates for the endogenous enzymes which synthesize nitric oxide.

Suitable NONOates include, but are not limited to, (Z)-1-(N-methyl-N-(6-(N-methyl-ammoniohexyl)amino))diazen-1-ium-1,2-diolate (“MAHMA/NO”), (Z)-1-(N-(3-ammoniopropyl)-N-(n-propyl)amino)diazen-1-ium-1,2-diolate (“PAPA/NO”), (Z)-1-(N-(3-aminopropyl)-N-(4-(3-aminopropylammonio)butyl)-amino) diazen-1-ium-1,2-diolate (spermine NONOate or “SPER/NO”) and sodium(Z)-1-(N,N-diethylamino)diazenium-1,2-diolate (diethylamine NONOate or “DEA/NO”) and derivatives thereof. NONOates are also described in U.S. Pat. Nos. 6,232,336, 5,910,316 and 5,650,447, the disclosures of which are incorporated herein by reference in their entirety. The “NO adducts” can be mono-nitrosylated, poly-nitrosylated, mono-nitrosated and/or poly-nitrosated at a variety of naturally susceptible or artificially provided binding sites for biologically active forms of nitrogen monoxide.

Suitable furoxanes include, but are not limited to, CAS 1609, C93-4759, C92-4678, S35b, CHF 2206, CHF 2363, and the like.

Suitable sydnonimines include, but are not limited to, molsidomine (N-ethoxycarbonyl-3-morpholinosydnonimine), SIN-1 (3-morpholinosydnonimine) CAS 936 (3-(cis-2,6-dimethylpiperidino)-N-(4-methoxybenzoyl)-sydnonimine, pirsidomine), C87-3754 (3-(cis-2,6-dimethylpiperidino)sydnonimine, linsidomine, C4144 (3-(3,3-dimethyl-1,4-thiazane-4-yl)sydnonimine hydrochloride), C89-4095 (3-(3,3-dimethyl-1,1-dioxo-1,4-thiazane-4-yl)sydnonimine hydrochloride, and the like.

Suitable oximes, include, but are not limited to, NOR-1, NOR-3, NOR-4, and the like.

One group of nitric oxide donor compounds is the S-nitrosothiols, which are compounds that include at least one —S—NO group. These compounds include S-nitroso-polypeptides (the term “polypeptide” includes proteins and polyamino acids that do not possess an ascertained biological function, and derivatives thereof); S-nitrosylated amino acids (including natural and synthetic amino acids and their stereoisomers and racemic mixtures and derivatives thereof); S-nitrosylated sugars; S-nitrosylated, modified and unmodified, oligonucleotides (preferably of at least 5, and more preferably 5-200 nucleotides); straight or branched, saturated or unsaturated, aliphatic or aromatic, substituted or unsubstituted S-nitrosylated hydrocarbons; and S-nitroso heterocyclic compounds. S-nitrosothiols and methods for preparing them are described in U.S. Pat. Nos. 5,380,758 and 5,703,073; WO 97/27749; WO 98/19672; and Oae et al, Org. Prep. Proc. Int., 15(3):165-198 (1983), the disclosures of each of which are incorporated by reference herein in their entirety.

Another embodiment of the invention is S-nitroso amino acids where the nitroso group is linked to a sulfur group of a sulfur-containing amino acid or derivative thereof. Such compounds include, for example, S-nitroso-N-acetylcysteine, S-nitroso-captopril, S-nitroso-N-acetylpenicillamine, S-nitroso-homocysteine, S-nitroso-cysteine, S-nitroso-glutathione, S-nitroso-cysteinyl-glycine, and the like.

Suitable S-nitrosylated proteins include thiol-containing proteins (where the NO group is attached to one or more sulfur groups on an amino acid or amino acid derivative thereof) from various functional classes including enzymes, such as tissue-type plasminogen activator (TPA) and cathepsin B; transport proteins, such as lipoproteins; heme proteins, such as hemoglobin and serum albumin; and biologically protective proteins, such as immunoglobulins, antibodies and cytokines. Such nitrosylated proteins are described in WO 93/09806, the disclosure of which is incorporated by reference herein in its entirety. Examples include polynitrosylated albumin where one or more thiol or other nucleophilic centers in the protein are modified.

Other examples of suitable S-nitrosothiols include:

HS(C(Rₑ)(R_{f}))ₘSNO; (i)

ONS(C(Rₑ)(R_{f}))ₘRₑ; or (ii)

H₂N—CH(CO₂H)—(CH₂)ₘ—C(O)NH—CH(CH₂SNO)—C(O)NH—CH₂—CO₂H; (iii)

wherein m is an integer from 2 to 20;

Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, an arylalklythio, an arylalklythioalkyl, an alkylthioalkyl, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano, an aminoalkyl, an aminoaryl, an aryl, an arylalkyl, an alkylaryl, a carboxamido, an alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, —U₃—V₅, V₆, —(C(Rₒ)(Rₚ))ₖ₁—U₃—V₅, —(C(Rₒ)(Rₚ))ₖ₁—U₃—V₆, —(C(Rₒ)(Rₚ))ₖ₁—U₃—C(O)—V₆, or Rₑ and R_{f} taken together with the carbons to which they are attached form a carbonyl, a methanthial, a heterocyclic ring, a cycloalkyl group, an aryl group, an oxime, a hydrazone, a bridged cycloalkyl group,

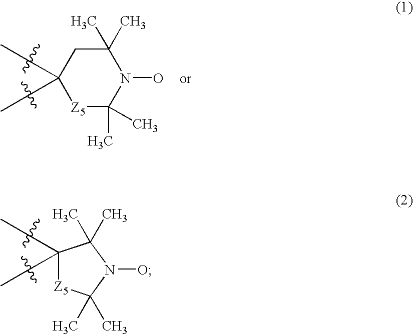

Rₒ and Rₚ are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, an arylalklythio, an arylalklythioalkyl, an alkylthioalkyl a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano, an aminoalkyl, an aminoaryl, an aryl, an arylalkyl, an alkylaryl, a carboxamido, an alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, —U₃—V₅, V₆, or Rₒ and Rₚ taken together with the carbons to which they are attached form a carbonyl, a methanthial, a heterocyclic ring, a cycloalkyl group, an aryl group, an oxime, an imine, a hydrazone, a bridged cycloalkyl group,

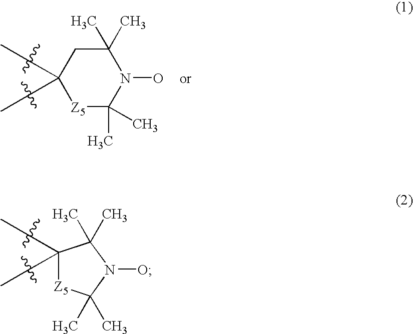

Z₅ is —CH₂ or oxygen;

k₁ is an integer form 1 to 3;
U₃ is an oxygen, sulfur- or —N(Rₐ)Rᵢ;

V₅ is —NO or —NO₂ (i.e. an oxidized nitrogen);

Rₐ is a lone pair of electrons, a hydrogen or an alkyl group;

Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an aminoalkyl, an aminoaryl, —CH₂—C(U₃—V₅)(Rₑ)(R_{f}), a bond to an adjacent atom creating a double bond to that atom or —(N₂O₂—)⁻.M₁⁺, wherein M₁⁺ is an organic or inorganic cation.

In cases where Rₑ and R_{f} are independently a heterocyclic ring or taken together Rₑ and R_{f} are a heterocyclic ring, then Rᵢ can be a substituent on any disubstituted nitrogen contained within the radical wherein Rᵢ is as defined herein.

Nitrosothiols can be prepared by various methods of synthesis. In general, the thiol precursor is prepared first, then converted to the S-nitrosothiol derivative by nitrosation of the thiol group with NaNO₂ under acidic conditions (pH is about 2.5) which yields the S-nitroso derivative. Acids which can be used for this purpose include aqueous sulfuric, acetic and hydrochloric acids. The thiol precursor can also be nitrosylated by reaction with an organic nitrite such as tert-butyl nitrite, or a nitrosonium salt such as nitrosonium tetrafluoroborate in an inert solvent.

Another group of nitric oxide donor compounds for use in the invention, where the nitric oxide donor is a compound that donates, transfers or releases nitric oxide, include compounds comprising at least one ON—O— or ON—N— group. The compounds that include at least one ON—O— or ON—N— group are ON—O— or ON—N-polypeptides (the term “polypeptide” includes proteins and polyamino acids that do not possess an ascertained biological function, and derivatives thereof); ON—O— or ON—N-amino acids (including natural and synthetic amino acids and their stereoisomers and racemic mixtures); ON—O— or ON—N-sugars; ON—O— or —ON—N— modified or unmodified oligonucleotides (comprising at least 5 nucleotides, preferably 5-200 nucleotides); ON—O— or ON—N— straight or branched, saturated or unsaturated, aliphatic or aromatic, substituted or unsubstituted hydrocarbons; and ON—O—, ON—N— or ON—C-heterocyclic compounds. Examples of compounds comprising at least one ON—O— or ON—N— group include butyl nitrite, isobutyl nitrite, tert-butyl nitrite, amyl nitrite, isoamyl nitrite, N-nitrosamines, N-nitrosamides, N-nitrosourea, N-nitrosoguanidines, N-nitrosocarbamates, N-acyl-N-nitroso compounds (such as, N-methyl-N-nitrosourea); N-hydroxy-N-nitrosamines, cupferron, alanosine, dopastin, 1,3-disubstituted nitrosiminobenzimidazoles, 1,3,4-thiadiazole-2-nitrosimines, benzothiazole-2(3H)-nitrosimines, thiazole-2-nitrosimines, oligonitroso sydnonimines, 3-alkyl-N-nitroso-sydnonimines, 2H-1,3,4-thiadiazine nitrosimines.

Another group of nitric oxide donor compounds for use in the invention include nitrates that donate, transfer or release nitric oxide, such as compounds comprising at least one O₂N—O—, O₂N—N— or O₂N—S— group. Among these compounds are O₂N—O—, O₂N—N— or O₂N—S— polypeptides (the term “polypeptide” includes proteins and also polyamino acids that do not possess an ascertained biological function, and derivatives thereof); O₂N—O—, O₂N—N— or O₂N—S— amino acids (including natural and synthetic amino acids and their stereoisomers and racemic mixtures); O₂N—O—, O₂N—N— or O₂N—S— sugars; O₂N—O—, O₂N—N— or O₂N—S-modified and unmodified oligonucleotides (comprising at least 5 nucleotides, preferably 5-200 nucleotides); O₂N—O—, O₂N—N— or O₂N—S— straight or branched, saturated or unsaturated, aliphatic or aromatic, substituted or unsubstituted hydrocarbons; and O₂N—O—, O₂N—N— or O₂N—S— heterocyclic compounds. Examples of compounds comprising at least one O₂N—O—, O₂N—N— or O₂N—S— group include isosorbide dinitrate, isosorbide mononitrate, clonitrate, erythrityl tetranitrate, mannitol hexanitrate, nitroglycerin, pentaerytlritoltetranitrate, pentrinitrol, propatylnitrate and organic nitrates with a sulfhydryl-containing amino acid such as, for example SPM 3672, SPM 4757, SPM 5185, SPM 5186 and those disclosed in U.S. Pat. Nos. 5,284,872, 5,428,061, 5,661,129, 5,807,847 and 5,883,122 and in WO 97/46521, WO 00/54756 and in WO 03/013432, the disclosures of each of which are incorporated by reference herein in their entirety.

Another group of nitric oxide donor compounds are N-oxo-N-nitrosoamines that donate, transfer or release nitric oxide and are represented by the formula: R^{1″}R^{2″}N—N(O-M⁺)-NO, where R^{1″} and R^{2″} are each independently a polypeptide, an amino acid, a sugar, a modified or unmodified oligonucleotide, a straight or branched, saturated or unsaturated, aliphatic or aromatic, substituted or unsubstituted hydrocarbon, or a heterocyclic group, and where M₁⁺ is an organic or inorganic cation, such, as for example, an alkyl substituted ammonium cation or a Group I metal cation.

The invention is also directed to compounds that stimulate endogenous NO or elevate levels of endogenous endothelium-derived relaxing factor (EDRF) in vivo or are oxidized to produce nitric oxide and/or are substrates for nitric oxide synthase and/or cytochrome P450. Such compounds include, for example, L-arginine, L-homoarginine, and N-hydroxy-L-arginine, N-hydroxy-L-homoarginine, N-hydroxydebrisoquine, N-hydroxypentamidine including their nitrosated and/or nitrosylated analogs (e.g., nitrosated L-arginine, nitrosylated L-arginine, nitrosated N-hydroxy-L-arginine, nitrosylated N-hydroxy-L-arginine, nitrosated and nitrosylated L-homoarginine), N-hydroxyguanidine compounds, amidoxime, ketoximes, aldoxime compounds, that can be oxidized in vivo to produce nitric oxide. Compounds that may be substrates for a cytochrome P450, include, for example, imino(benzylamino)methylhydroxylamine, imino(((4-methylphenyl)methyl) amino)methylhydroxylamine, imino(((4-methoxyphenyl)methyl)amino) methylhydroxylamine, imino(((4-(trifluoromethyl)phenyl)methyl) amino) methylhydroxylamine, imino(((4-nitrophenyl) methyl)amino)methylhydroxylamine, (butylamino)iminomethylhydroxylamine, imino(propylamino)methylhydroxylamine, imino(pentylamino)methylhydroxylamine, imino(propylamino)methylhydroxylamine, imino ((methylethyl)amino)methylhydroxylamine, (cyclopropylamino)iminomethylhydroxylamine, imino-2-1,2,3,4-tetrahydroisoquinolyl methylhydroxylamine, imino(1-methyl(2-1,2,3,4-tetrahydroisoquinolyl))methylhydroxylamine, (1,3-dimethyl(2-1,2,3,4-tetrahydroisoquinolyl)) iminomethylhydroxylamine, (((4-chlorophenyl)methyl) amino)iminomethylhydroxylamine, ((4-chlorophenyl)amino) iminomethylhydroxylamine, (4-chlorophenyl)(hydroxyimino) methylamine, and 1-(4-chlorophenyl)-1-(hydroxyimino) ethane, and the like, precursors of L-arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid), nitric oxide mediators and/or physiologically acceptable salts thereof, including, for example, pyruvate, pyruvate precursors, α-keto acids having four or more carbon atoms, precursors of α-keto acids having four or more carbon atoms (as disclosed in WO 03/017996, the disclosure of which is incorporated herein in its entirety), and the substrates for nitric oxide synthase, cytokines, adenosin, bradykinin, calreticulin, bisacodyl, and phenolphthalein. EDRF is a vascular relaxing factor secreted by the endothelium, and has been identified as nitric oxide (NO) or a closely related derivative thereof (Palmer et al, Nature, 327:524-526 (1987); Ignarro et al, Proc. Natl. Acad. Sci. USA, 84:9265-9269 (1987)).

The invention is also directed to nitric oxide enhancing compounds that can increase endogenous nitric oxide. Such compounds, include for example, nitroxide containing compounds that include, but are not limited to, substituted 2,2,6,6-tetramethyl-1-piperidinyloxy compounds, substituted 2,2,5,5-tetramethyl-3-pyrroline-1-oxyl compounds, substituted 2,2,5,5-tetramethyl-1-pyrrolidinyloxyl compounds, substituted 1,1,3,3-tetramethylisoindolin-2-yloxyl compounds, substituted 2,2,4,4-tetramethyl-1-oxazolidinyl-3-oxyl compounds, substituted 3-imidazolin-1-yloxy, 2,2,5,5-tetramethyl-3-imidazolin-1-yloxyl compounds, OT-551, 4-hydroxy-2,2,6,6-tetramethyl-1-piperidinyloxy (tempol), and the like. Suitable substituents include, but are not limited to, aminomethyl, benzoyl, 2-bromoacetamido, 2-(2-(2-bromoacetamido)ethoxy)ethylcarbamoyl, carbamoyl, carboxy, cyano, 5-(dimethylamino)-1-naphthalenesulfonamido, ethoxyfluorophosphinyloxy, ethyl, 5-fluoro-2,4-dinitroanilino, hydroxy, 2-iodoacetamido, isothiocyanato, isothiocyanatomethyl, methyl, maleimido, maleimidoethyl, 2-(2-maleimidoethoxy)ethylcarbamoyl, maleimidomethyl, maleimido, oxo, phosphonooxy, and the like.

In one embodiment of the invention the nitric oxide enhancing compound is isosorbide dinitrate and/or isosorbide mononitrate.

In one embodiment the hydralazine compound is hydralazine, which is can be administered in the form of a pharmaceutically acceptable salt. In another embodiment the pharmaceutically acceptable salt of the hydralazine compound is hydralazine hydrochloride. Hydralazine hydrochloride is commercially available from, for example, Lederle Standard Products, Pearl River, N.Y.; and Par Pharmaceuticals Inc., Spring Valley, N.Y. It is a white to off-white, crystalline powder and is soluble in water, slightly soluble in alcohol and very slightly soluble in ether. The hydralazine compound can be stabilized to prevent degradation by the addition of chelating agents, such as, for example, ethylenediamine tetracidic acid, citric acid, fumeric acid, and the like.

Isosorbide dinitrate is commercially available, for example, under the trade names DILATRATE®-SR (Schwarz Pharma, Milwaukee, Wis.); ISORDIL® and ISORDILR TITRADOSE® (Wyeth Laboratories Inc., Philadelphia, Pa.); and SORBITRATE® (Zeneca Pharmaceuticals, Wilmington, Del.). Diluted isosorbide dinitrate (1,4,3,6-dianhydro-D-glucitol-2,5-dinitrate), USP, is a white to off-white powder. It is freely soluble in organic solvents such as ethanol, ether and chloroform, but is sparingly soluble in water.

Isosorbide mononitrate is commercially available, for example, under the trade names IMDUR® (A. B. Astra, Sweden); MONOKET® (Schwarz Pharma, Milwaukee, Wis.); and ISMO® (Wyeth-Ayerst Company, Philadelphia, Pa.).

The isosorbide dinitrate and isosorbide mononitrate can be stabilized to prevent explosions by the addition of compounds, such as, but not limited to, lactose, arginine, mannitol, sorbitol, cellulose (Avicel®) and the like, and combinations of two or more thereof.

The hydralazine compound and at least one of isosorbide dinitrate and isosorbide mononitrate can be administered as separate components or as components of the same composition. When the hydralazine compound and at least one of isosorbide dinitrate and isosorbide mononitrate are administered as separate components, can be administered to the patient at about the same time. “About the same time” means that within about thirty minutes of administering one compound (e.g., the hydralazine compound or isosorbide dinitrate/mononitrate) to the patient, the other compound (e.g., isosorbide dinitrate/mononitrate or the hydralazine compound) is administered to the patient. “About the same time” also includes simultaneous administration of the compounds.

The invention provides methods for reducing mortality associated with heart failure; improving oxygen consumption; treating heart failure; treating hypertension; improving the quality of life in a heart failure patient; inhibiting left ventricular remodeling; reducing hospitalizations related to heart failure; improving exercise tolerance; increasing left ventricular ejection fraction; decreasing levels of B-type natriuretic protein; in a patient in need thereof, wherein the patient has a −344 (T/T) polymorphism or a −344 (C/C) polymorphism in an aldosterone synthase CYP11B2 gene, comprising administering to the patient an effective amount of at (i) at least one antioxidant compound or pharmaceutically acceptable salt thereof; (ii) at least one nitric oxide enhancing compound; and (iii) optionally at least one compound selected from the group consisting of an angiotensin converting enzyme inhibitor, a β-adrenergic antagonist, an angiotensin II antagonist, an aldosterone antagonist, a cardiac glycoside and a diuretic compound or a combination of two or more thereof. In one embodiment the antioxidant is a hydralazine compound or a pharmaceutically acceptable salt thereof and the nitric oxide enhancing compound is isosorbide dinitrate and/or isosorbide mononitrate. In these embodiments, the methods can involve (i) administering the hydralazine compound or a pharmaceutically acceptable salt thereof, and at least one of isosorbide dinitrate and/or isosorbide mononitrate, or (ii) administering the hydralazine compound or a pharmaceutically acceptable salt thereof, at least one of isosorbide dinitrate and/or isosorbide mononitrate, and at least one compound selected from the group consisting of an angiotensin converting enzyme inhibitor, a β-adrenergic antagonist, an angiotensin II antagonist, an aldosterone antagonist, a cardiac glycoside and a diuretic compound or a combination of two or more thereof. In yet another embodiment the hydralazine compound or a pharmaceutically acceptable salt thereof is hydralazine hydrochloride. In another embodiment the patient has at least one polymorphism in the endothelial nitric oxide synthase (NOS3) gene and/or at least one polymorphism in the beta 1 adrenergic receptor gene. In these embodiments the at least one polymorphism in the endothelial nitric oxide synthase (NOS3) gene is an Asp298Glu polymorphism in exon 7 of the endothelial nitric oxide synthase gene, a T-786C polymorphism in the promoter region of the endothelial nitric oxide synthase gene and/or a 27 base-pair tandem repeat intron 4 polymorphism of the endothelial nitric oxide synthase gene and the at least one polymorphism in the beta 1 adrenergic receptor gene is a Arg389Arg polymorphism and/or a Gly389Gly polymorphism in the beta 1 adrenergic receptor gene. In these embodiments, the Asp298Glu polymorphism in exon 7 of the endothelial nitric oxide synthase gene is a Glu298Glu variant; the T-786C polymorphism in the promoter region of the endothelial nitric oxide synthase gene is a T-786C variant or a T-786T variant; and the intron 4 polymorphism in the endothelial nitric oxide synthase gene is an intron 4a/4b variant or an intron 4b/4b variant. In another embodiment, the patient is categorized as New York Heart Association heart failure functional classification I, II, III or IV. In yet another embodiment, the patient is categorized as New York Heart Association heart failure functional classification II, III or IV. In yet another embodiment the patient is a black patient. The hydralazine compounds, isosorbide dinitrate and/or isosorbide mononitrate and/or additional compounds can be administered separately or as components of the same composition in one or more pharmaceutically acceptable carriers.

The invention provides treating renovascular diseases; treating end-stage renal diseases; reducing cardiomegaly; treating diseases resulting from oxidative stress; treating endothelial dysfunctions; treating diseases caused by endothelial dysfunctions; treating cardiovascular diseases; in a patient in need thereof, wherein the patient has a −344 (T/T) polymorphism or a −344 (C/C) polymorphism in an aldosterone synthase CYP11B2 gene, comprising administering to the patient an effective amount of at (i) at least one antioxidant compound or pharmaceutically acceptable salt thereof; (ii) at least one nitric oxide enhancing compound; and (iii) optionally at least one compound selected from the group consisting of an angiotensin converting enzyme inhibitor, a β-adrenergic antagonist, an angiotensin II antagonist, an aldosterone antagonist, a cardiac glycoside and a diuretic compound or a combination of two or more thereof. In one embodiment the antioxidant is a hydralazine compound or a pharmaceutically acceptable salt thereof and the nitric oxide enhancing compound is isosorbide dinitrate and/or isosorbide mononitrate. In these embodiments, the methods can involve (i) administering the hydralazine compound or a pharmaceutically acceptable salt thereof, and at least one of isosorbide dinitrate and/or isosorbide mononitrate, or (ii) administering the hydralazine compound or a pharmaceutically acceptable salt thereof, at least one of isosorbide dinitrate and/or isosorbide mononitrate, and at least one compound selected from the group consisting of an angiotensin converting enzyme inhibitor, a β-adrenergic antagonist, an angiotensin II antagonist, an aldosterone antagonist, a cardiac glycoside and a diuretic compound or a combination of two or more thereof. In yet another embodiment the hydralazine compound or a pharmaceutically acceptable salt thereof is hydralazine hydrochloride. In another embodiment the patient has at least one polymorphism in the endothelial nitric oxide synthase (NOS3) gene and/or at least one polymorphism in the beta 1 adrenergic receptor gene. In these embodiments the at least one polymorphism in the endothelial nitric oxide synthase (NOS3) gene is an Asp298Glu polymorphism in exon 7 of the endothelial nitric oxide synthase gene, a T-786C polymorphism in the promoter region of the endothelial nitric oxide synthase gene and/or a 27 base-pair tandem repeat intron 4 polymorphism of the endothelial nitric oxide synthase gene and the at least one polymorphism in the beta 1 adrenergic receptor gene is a Arg389Arg polymorphism and/or a Gly389Gly polymorphism in the beta 1 adrenergic receptor gene. In these embodiments, the Asp298Glu polymorphism in exon 7 of the endothelial nitric oxide synthase gene is a Glu298Glu variant; the T-786C polymorphism in the promoter region of the endothelial nitric oxide synthase gene is a T-786C variant or a T-786T variant; and the intron 4 polymorphism in the endothelial nitric oxide synthase gene is an intron 4a/4b variant or an intron 4b/4b variant. In another embodiment, the patient is categorized as New York Heart Association heart failure functional classification I, II, III or IV; preferably II, III or IV. In yet another embodiment the patient is a black patient. The hydralazine compounds, isosorbide dinitrate and/or isosorbide mononitrate and/or additional compounds can be administered separately or as components of the same composition in one or more pharmaceutically acceptable carriers.

In another embodiment, the invention provides methods of administering (i) a hydralazine compound (e.g., hydralazine hydrochloride), (ii) isosorbide dinitrate and/or isosorbide mononitrate (e.g., isosorbide dinitrate), and (iii) an aldosterone antagonist. In another embodiment, the invention provides methods of administering (i) a hydralazine compound (e.g., hydralazine hydrochloride), (ii) isosorbide dinitrate and/or isosorbide mononitrate (e.g., isosorbide dinitrate), and (iii) an angiotensin converting enzyme inhibitor. In another embodiment, the invention provides methods of administering (i) a hydralazine compound (e.g., hydralazine hydrochloride), (ii) isosorbide dinitrate and/or isosorbide mononitrate (e.g., isosorbide dinitrate), and (iii) a β-adrenergic antagonist. In another embodiment, the invention provides methods of administering (i) a hydralazine compound (e.g., hydralazine hydrochloride), (ii) isosorbide dinitrate and/or isosorbide mononitrate (e.g., isosorbide dinitrate), and (iii) an angiotensin II antagonist. In another embodiment, the invention provides methods of administering (i) a hydralazine compound (e.g., hydralazine hydrochloride), (ii) isosorbide dinitrate and/or isosorbide mononitrate (e.g., isosorbide dinitrate), and (iii) a digitalis. In another embodiment, the invention provides methods of administering (i) a hydralazine compound (e.g., hydralazine hydrochloride), (ii) isosorbide dinitrate and/or isosorbide mononitrate (e.g., isosorbide dinitrate), and (iii) a diuretic compound. In another embodiment, the invention provides methods of administering (i) a hydralazine compound (e.g., hydralazine hydrochloride), (ii) isosorbide dinitrate and/or isosorbide mononitrate (e.g., isosorbide dinitrate), (iii) an angiotensin converting enzyme inhibitor, and (iv) a β-adrenergic antagonist. In another embodiment, the invention provides methods of administering (i) a hydralazine compound (e.g., hydralazine hydrochloride), (ii) isosorbide dinitrate and/or isosorbide mononitrate (e.g., isosorbide dinitrate), (iii) an angiotensin converting enzyme inhibitor, and (iv) an angiotensin II antagonist. In another embodiment, the invention provides methods of administering (i) a hydralazine compound (e.g., hydralazine hydrochloride), (ii) isosorbide dinitrate and/or isosorbide mononitrate (e.g., isosorbide dinitrate), (iii) an angiotensin converting enzyme inhibitor, and (iv) an aldosterone antagonist. In another embodiment, the invention provides methods of administering (i) a hydralazine compound (such as, hydralazine hydrochloride), (ii) isosorbide dinitrate and/or isosorbide mononitrate (such as, isosorbide dinitrate), (iii) an angiotensin converting enzyme inhibitor, and (iv) a diuretic. In another embodiment, the invention provides methods of administering (i) a hydralazine compound (such as, hydralazine hydrochloride), (ii) isosorbide dinitrate and/or isosorbide mononitrate (such as, isosorbide dinitrate), (iii) a β-adrenergic antagonist, and (iv) an angiotensin II antagonist. In another embodiment, the invention provides methods of administering (i) a hydralazine compound (such as, hydralazine hydrochloride), (ii) isosorbide dinitrate and/or isosorbide mononitrate (such as, isosorbide dinitrate), (iii) a β-adrenergic antagonist, and (iv) an aldosterone antagonist. In another embodiment, the invention provides methods of administering (i) a hydralazine compound (such as, hydralazine hydrochloride), (ii) isosorbide dinitrate and/or isosorbide mononitrate (such as, isosorbide dinitrate), (iii) a β-adrenergic antagonist, and (iv) a diuretic. In another embodiment, the invention provides methods of administering (i) a hydralazine compound (such as, hydralazine hydrochloride), (ii) isosorbide dinitrate and/or isosorbide mononitrate (such as, isosorbide dinitrate), (iii) an angiotensin II antagonist and (iv) an aldosterone antagonist. In another embodiment, the invention provides methods of administering (i) a hydralazine compound (such as, hydralazine hydrochloride), (ii) isosorbide dinitrate and/or isosorbide mononitrate (such as, isosorbide dinitrate), (iii) an angiotensin II antagonist and (iv) a diuretic. In another embodiment, the invention provides methods of administering (i) a hydralazine compound (such as, hydralazine hydrochloride), (ii) isosorbide dinitrate and/or isosorbide mononitrate (such as, isosorbide dinitrate), (iii) an aldosterone antagonist and (iv) a diuretic. In another embodiment, the invention provides methods of administering (i) a hydralazine compound (such as, hydralazine hydrochloride), (ii) isosorbide dinitrate and/or isosorbide mononitrate (such as, isosorbide dinitrate), (iii) an angiotensin converting enzyme inhibitor, (iv) a β-adrenergic antagonist, and (v) an aldosterone antagonist. In another embodiment, the invention provides methods of administering (i) a hydralazine compound (such as, hydralazine hydrochloride), (ii) isosorbide dinitrate and/or isosorbide mononitrate (such as, isosorbide dinitrate), (iii) an angiotensin converting enzyme inhibitor, (iv) a β-adrenergic antagonist, and (v) an angiotensin II antagonist. In another embodiment, the invention provides methods of administering (i) a hydralazine compound (such as, hydralazine hydrochloride), (ii) isosorbide dinitrate and/or isosorbide mononitrate (such as, isosorbide dinitrate), (iii) a diuretic compound, and (iv) a cardiac glycoside. In these embodiments the hydralazine compound, and at least one of isosorbide dinitrate and isosorbide mononitrate can be administered separately or as components of the same composition, and can be administered in the form of a composition with or simultaneously with, subsequently to, or prior to administration of at least one of the angiotensin converting enzyme inhibitor, β-adrenergic antagonist, angiotensin II antagonist, aldosterone antagonist, digitalis, diuretic compound or combinations of two or more thereof. In one embodiment, all the compounds are administered together in the form of a single composition.

In one embodiment, the hydralazine hydrochloride can be administered in an amount of about 30 milligrams per day to about 400 milligrams per day; the isosorbide dinitrate can be administered in an amount of about 10 milligrams per day to about 200 milligrams per day; or the isosorbide mononitrate can be administered in an amount of about 5 milligrams per day to about 120 milligrams per day. In another embodiment, the hydralazine hydrochloride can be administered in an amount of about 50 milligrams per day to about 300 milligrams per day; the isosorbide dinitrate can be administered in an amount of about 20 milligrams per day to about 160 milligrams per day; or the isosorbide mononitrate can be administered in an amount of about 15 milligrams per day to about 100 milligrams per day. In another embodiment, the hydralazine hydrochloride can be administered in an amount of about 37.5 milligrams to about 75 milligrams one to four times per day; the isosorbide dinitrate can be administered in an amount of about 20 milligrams to about 40 milligrams one to four times per day; or the isosorbide mononitrate can be administered in an amount of about 10 milligrams to about 20 milligrams one to four times per day. The particular amounts of hydralazine and isosorbide dinitrate or isosorbide mononitrate can be administered as a single dose once a day; in multiple doses several times throughout the day; as a sustained-release oral formulation; as an injectable formulation; or as an inhalation formulation.

In one embodiment of the methods of the invention, the patient can be administered a composition comprising about 225 mg hydralazine hydrochloride and about 120 mg isosorbide dinitrate once per day (i.e., q.d.). In another embodiment of the methods of the invention, the patient can be administered a composition comprising about 112.5 mg hydralazine hydrochloride and about 60 mg isosorbide dinitrate twice per day (i.e., b.i.d.). In another embodiment of the methods of the invention, the patient can be administered a composition comprising about 56.25 mg hydralazine hydrochloride and about 30 mg isosorbide dinitrate twice per day (i.e., b.i.d.). In another embodiment, the patient can be administered a composition comprising about 75 mg hydralazine hydrochloride and about 40 mg isosorbide dinitrate three times per day (i.e., t.i.d.). In another embodiment of the methods of the invention, the patient can be administered a composition comprising about 37.5 mg hydralazine hydrochloride and about 20 mg isosorbide dinitrate three times per day (i.e., t.i.d.). The particular amounts of hydralazine and isosorbide dinitrate or isosorbide mononitrate can be administered as a sustained-release oral formulation; as an injectable formulation; or as an inhalation formulation.

In any of the embodiments described herein, the patient can be administered one, two or three compositions (e.g., two tablets, two capsules, two injections, and the like) at any particular time. For example, the patient can be administered two separate compositions, wherein each composition comprises about 112.5 mg hydralazine hydrochloride and about 60 mg isosorbide dinitrate twice per day (i.e., b.i.d.). In another embodiment, the patient can be administered two separate compositions, wherein each composition comprises about 56.25 mg hydralazine hydrochloride and about 30 mg isosorbide dinitrate twice per day (i.e., b.i.d.).

In the invention the at least one hydralazine compound or pharmaceutically acceptable salts thereof, and at least one of isosorbide dinitrate and isosorbide mononitrate, are administered as separate components or as components of the same composition with at least one of the angiotensin converting enzyme inhibitor, β-adrenergic antagonist, angiotensin II antagonist, aldosterone antagonist, cardiac glycoside, diuretic compound or a combination of two or more thereof. They can also be administered as separate components as single doses once a day; or in multiple doses several times throughout the day; or as a sustained-release oral formulation; or as an injectable formulation.

In one embodiment, the invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) optionally an angiotensin-converting enzyme inhibitor. Suitable angiotensin-converting enzyme inhibitors (ACE inhibitors) include, but are not limited to, alacepril, benazepril (LOTENSIN®, CIBACEN8), benazeprilat, captopril, ceronapril, cilazapril, delapril, duinapril, enalapril, enalaprilat, fasidotril, fosinopril, fosinoprilat, gemopatrilat, glycopril, idrapril, imidapril, lisinopril, moexipril, moveltipril, naphthopidil, omapatrilat, pentopril, perindopril, perindoprilat, quinapril, quinaprilat, ramipril, ramiprilat, rentipril, saralasin acetate, spirapril, temocapril, trandolapril, trandolaprilat, urapidil, zofenopril, acylmercapto and mercaptoalkanoyl pralines, carboxyalkyl dipeptides, carboxyalkyl dipeptide, phosphinylalkanoyl pralines, registry no. 796406, AVE 7688, BP1.137, CHF 1514, E 4030, ER 3295, FPL-66564, MDL 100240, RL 6134, RL 6207, RL 6893, SA 760, S-5590, Z 13752A, and the like. One skilled in the art will appreciate that the angiotensin-converting enzyme inhibitors may be administered in the form of pharmaceutically acceptable salts, hydrates, acids and/or stereoisomers thereof. Suitable angiotensin-converting enzyme inhibitors are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996; and on STN Express, file phar and file registry.

In some embodiments the angiotensin-converting enzyme inhibitors are benazepril, captopril, enalapril, fosinopril, lisinopril, moexipril, quinapril, ramipril, trandolapril or trandolaprilat. In other embodiments the benazepril is administered as benazepril hydrochloride in an amount of about 5 milligrams to about 80 milligrams as a single dose or as multiple doses per day; the captopril is administered in an amount of about 12.5 milligrams to about 450 milligrams as a single dose or as multiple doses per day; the enalapril is administered as enalapril maleate in an amount of about 2.5 milligrams to about 40 milligrams as a single dose or as multiple doses per day; the fosinopril is administered as fosinopril sodium in an amount of about 5 milligrams to about 60 milligrams as a single dose or as multiple doses per day; the lisinopril is administered in an amount of about 2.5 milligrams to about 75 milligrams as a single dose or as multiple doses per day; the moexipril is administered as moexipril hydrochloride in an amount of about 7.5 milligrams to about 45 milligrams as a single dose or as multiple doses per day; the quinapril is administered as quinapril hydrochloride in an amount of about 5 milligrams to about 40 milligrams as single or multiple doses per day; the ramipril hydrochloride is administered in an amount of about 1.25 milligrams to about 40 milligrams as single or multiple doses per day; the trandolapril is administered in an amount of about 0.5 milligrams to about 4 milligrams as single or multiple doses per day; the trandolaprilat is administered in an amount of about 0.5 milligrams to about 4 milligrams as single or multiple doses per day. In other embodiments the angiotensin-converting enzyme inhibitors are captopril, enalapril, lisinopril, ramipril, trandolapril or trandolaprilat.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adienergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) captopril. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dimitrate and isosorbide mononitrate (e.g., isosorbide dinitrate), and (iii) enalapril. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) ramipril. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) lisinopril. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) trandolapril. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) trandolaprilat. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) a β-adrenergic antagonist. Suitable β-adrenergic antagonists include, but are not limited to, acebutolol, alprenolol, amosulalol, arotinolol, atenolol, befunolol, betaxolol, bevantolol, bisoprolol, bopindolol, bucindolol, bucumolol, bufetolol, bufuralol, bunitrolol, bupranolol, butofilolol, carazolol, capsinolol, carteolol, carvedilol (COREG®), celiprolol, cetamolol, cindolol, cloranolol, dilevalol, diprafenone, epanolol, ersentilide, esmolol, esprolol, hydroxalol, indenolol, labetalol, landiolol, laniolol, levobunolol, mepindolol, methylpranol, metindol, metipranolol, metrizoranolol, metoprolol, moprolol, nadolol, nadoxolol, nebivolol, nifenalol, nipradilol, oxprenolol, penbutolol, pindolol, practolol, pronethalol, propranolol, sotalol, sotalolnadolol, sulfinalol, taliprolol, talinolol, tertatolol, tilisolol, timolol, toliprolol, tomalolol, trimepranol, xamoterol, xibenolol, 2-(3-(1,1-dimethylethyl)-amino-2-hydroxypropoxy)-3-pyridenecarbonitrilHCl, 1-butylamino-3-(2,5-dichlorophenoxy)-2-propanol, 1-isopropylamino-3-(4-(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, 7-(2-hydroxy-3-t-butylaminpropoxy)phthalide, Acc 9369, AMO-140, BIB-16S, CP-331684, Fr-172516, ISV-208, L-653328, LM-2616, SB-226552, SR-58894A, SR-59230A, TZC-5665, UK-1745, YM-430, and the like. One skilled in the art will appreciate that the β-adrenergic antagonists can be administered in the form of pharmaceutically acceptable salts and/or stereoisomers. Suitable β-adrenergic antagonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In some embodiments the β-adrenergic antagonists are atenolol, bisoprolol, carvedilol, metoprolol, nebivolol, propranolol or timolol. In other embodiments the atenolol is administered in an amount of about 50 milligrams to about 200 milligrams as a single dose or as multiple doses per day; the bisoprolol is administered as bisoprolol fumarate in an amount of about 2.5 milligrams to about 30 milligrams as a single dose or as multiple doses per day; the carvedilol is administered in an amount of about 3.125 milligrams to about 200 milligrams as a single dose or as multiple doses per day; the metoprolol is administered as metoprolol tartarate or metoprolol succinate in an amount of about 25 milligrams to about 300 milligrams as a single dose or as multiple doses per day; the nebivolol is administered as nebivolol hydrochloride in an amount of about 2.5 milligrams to about 20 milligrams as a single dose or as multiple doses per day; the propranolol is administered as propranolol hydrochloride in an amount of about 40 milligrams to about 240 milligrams as a single dose or as multiple doses per day; the timolol is administered as timolol maleate in an amount of about 10 milligrams to about 30 milligrams as a single dose or as multiple doses per day. In other embodiments the β-adrenergic anagonists are bisoprolol, carvedilol, metoprolol or nebivolol.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) bisoprolol. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) carvedilol. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) metoprolol. The compounds can be administered separately or in the form of a composition.

The invention provides methods for (a) reducing mortality associated with heart failure; (b) improving oxygen consumption, (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) nebivolol. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) an angiotensin II antagonist Suitable angiotensin II antagonists include, but are not limited to, angiotensin, abitesartan, candesartan, candesartan cilexetil, elisartan, embusartan, enoltasosartan, eprosartan, fonsartan, forasartan, glycyllosartan, irbesartan, losartan, olmesartan, milfasartan, medoxomil, ripisartan, pomisartan, pratosartan, saprisartan, saralasin, sarmesin, tasosartan, telmisartan, valsartan, zolasartan, 3-(2′(tetrazole-5-yl)-1,1′-biphen-4-yl)methyl-5,7-dimethyl-2-ethyl-3H-imidazo[4,5-b]pyridine, antibodies to angiotensin II, A-81282, A-81988, BAY 106734, BIBR-363, BIBS-39, BIBS-222, BMS-180560, BMS-184698, BMS-346567, CGP-38560A, CGP-42112A, CGP-48369, CGP-49870, CGP-63170, CI-996, CP-148130, CL-329167, CV-11194, CV-11974, DA-2079, DE-3489, DMP-811, DuP-167, DuP-532, DuP-753, E-1477, E-4177, E-4188, EMD-66397, EMD-666R4, EMD-73495, EMD-66684, EXP-063, EXP-929, EXP-3134, EXP-3174, EXP-6155, EXP-6803, EXP-7711, EXP-9270, EXP-9954, FK-739, FRI 153332, GA-0050, GA-0056, HN-65021, HOE-720, HR-720, ICI-D6888, ICI-D7155, ICI-D8731, KRI-1177, KT3-671, KT-3579, KW-3433, L-158809, L-158978, L-159282 (MK-996), L-159689, L-159874, L-161177, L-162154, L-162234, L-162441, L-163007, L-163017, LF-70156, LRB-057, LRB-081, LRB-087, LY-235656, LY-266099, LY-285434, LY-301875, LY-302289, LY-315995, ME-3221, MK-954, PD-123177, PD-123319, PD-126055, PD-150304, RG-13647, RWJ-38970, RWJ-46458, S-8307, S-8308, SC-51757, SC-54629, SC-52458, SC-52459, SK 1080, SL-910102, SR-47436, TAK-536, UP-2696, U-96849, U-97018, UK-77778, UP-275-22, WAY-126227, WK-1260, WK-1360, WIC-1492, WY 126227, YH-1498, YM-358, YM-31472, X-6803, XH-148, XR-510, ZD-6888, ZD-7155, ZD-8731, ZD 8131, the compounds of ACS registry numbers 133240-46-7, 135070-05-2, 139958-16-0, 145160-84-5, 147403-03-0, 153806-29-2, 439904-54-8P, 439904-55-9P, 439904-56-0P, 439904-57-1P, 439904-58-2P, 155918-60-8P, 155918-61-9P, 272438-16-1P, 272446-75-0P, 223926-77-0P, 169281-89-4, 165113-17-7P, 165113-18-8P, 165113-19-9P, 165113-20-2P, 165113-13-3P, 165113-14-4P, 165113-15-5P, 165113-16-6P, 165113-21-3P, 165113-22-4P, 165113-23-5P, 165113-24-6P, 165113-25-7P, 165113-26-8P, 165113-27-9P, 165113-28-0P, 165113-29-1P, 165113-30-4P, 165113-31-5P, 165113-32-6P, 165113-33-7P, 165113-34-8P, 165113-35-9P, 165113-36-0P, 165113-37-1P, 165113-38-2P, 165113-39-3P, 165113-40-6P, 165113-41-7P, 165113-42-8P, 165113-43-9P, 165113-44-0P, 165113-45-1P, 165113-46-2P, 165113-47-3P, 165113-48-4P, 165113-49-5P, 165113-50-8P, 165113-51-9P, 165113-52-0P, 165113-53-1P, 165113-54-2P, 165113-55-3P, 165113-56-4P, 165113-57-5P, 165113-58-6P, 165113-59-7P, 165113-60-0P, 165113-61-1P, 165113-62-2P, 165113-63-3P, 165113-64-4P, 165113-65-5P, 165113-66-6P, 165113-67-7P, 165113-68-8P, 165113-69-9P, 165113-70-2P, 165113-71-3P, 165113-72-4P, 165113-73-5P, 165113-74-6P, 114798-27-5, 114798-28-6, 114798-29-7, 124749-82-2, 114798-28-6, 124749-84-4, 124750-88-5, 124750-91-0, 124750-93-2, 161946-65-2P, 161947-47-3P, 161947-48-4P, 161947-51-9P, 161947-52-0P, 161947-55-3P, 161947-56-4P, 161947-60-0P, 1619472-61-1P, 161947-68-8P, 161947-69-9P, 161947-70-2P, 161947-71-3P, 161947-72-4P, 161947-74-6P, 161947-75-7P, 161947-81-5P, 161947-82-6P, 161947-83-7P, 161947-84-8P, 161947-85-9P, 161947-86-0P, 161947-87-1P, 161947-88-2P, 161947-89-3P, 161947-90-6P, 161947-91-7P, 161947-92-8P, 161947-93-9P, 161947-94-0P, 161947-95-1P, 161947-96-2P, 161947-97-3P, 161947-98-4P, 161947-99-5P, 161948-00-1P, 161948-01-2P, 161948-02-3P, 168686-32-6P, 167301-42-0P, 166813-82-7P, 166961-56-4P, 166961-58-6P, 158872-96-9P, 158872-97-0P, 158807-14-8P, 158807-15-9P, 158807-16-0P, 158807-17-1P, 158807-18-2P, 158807-19-3P, 158807-20-6P, 155884-08-5P, 154749-99-2, 167371-59-7P, 244126-99-6P, 177848-35-0P, 141309-82-2P, and the like. Suitable angiotensin II antagonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In one embodiment the angiotensin II antagonists are candesartan, eprosartan, irbesartan, losartan, omlesartan, telmisartan or valsartan. In other embodiments the candesartan is administered as candesartan cilexetil in an amount of about 15 milligrams to about 100 milligrams as a single dose or as multiple doses per day; the eprosartan is administered as eprosartan mesylate in an amount of about 400 milligrams to about 1600 milligrams as a single dose or as multiple doses per day; the irbesartan is administered in an amount of about 75 milligrams to about 1200 milligrams as a single dose or as multiple doses per day; the losartan is administered as losartan potassium in an amount of about 25 milligrams to about 100 milligrams as a single dose or as multiple doses per day; the omlesartan is administered as omlesartan medoxomil in an amount of about 5 milligrams to about 40 milligrams as a single dose or as multiple doses per day; the telmisartan is administered in an amount of about 20 milligrams to about 80 milligrams as a single dose or as multiple doses per day; the valsartan is administered in an amount of about 80 milligrams to about 320 milligrams as a single dose or as multiple doses per day. In other embodiments the angiotensin II antagonists are candesartan, irbesartan, losartan or valsartan.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) candesartan. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) irbesartan. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) losartan. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) valsartan. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) an aldosterone antagonist. Suitable aldosterone antagonists include, but are not limited to, canrenone, potassium canrenoate, drospirenone, spironolactone, eplerenone (INSPRA®), epoxymexrenone, fadrozole, pregn-4-ene-7,21-dicarboxylic acid, 9,11-epoxy-17-hydroxy-3-oxo, γ-lactone, methyl ester, (7α,11α,17β)-; pregn-4-ene-7,21-dicarboxylic acid, 9,11-epoxy-17-hydroxy-3-oxo-dimethyl ester, (7α,11α,17β)-; 3′H-cyclopropa(6,7) pregna-4,6-diene-21-carboxylic acid, 9,11-epoxy-6,7-dihydro-17-hydroxy-3-oxo-, γ-lactone, (6β,7β,11α,17β)-; pregn-4-ene-7,21-dicarboxylic acid, 9,11-epoxy-17-hydroxy-3-oxo-, 7-(1-methylethyl) ester, monopotassium salt, (7α,11α,17β)-; pregn-4-ene-7,2,1-dicarboxylic acid, 9,11-epoxy-17-hydroxy-3-oxo-, 7-methyl ester, monopotassium salt, (7α,11α,7β)-; 3′H-cyclopropa(6,7) pregna-1,4,6-triene-21-carboxylic acid, 9,11-epoxy-6,7-dihydro-17-hydroxy-3-oxo-, γ-lactone, (6β,7β,11α)-; 3′H-cyclopropa(6,7)pregna-4,6-diene-21-carboxylic acid, 9,11-epoxy-6,7-dihydro-17-hydroxy-3-oxo-, methyl ester, (6β,7β,11α,17β)-; 3′H-cyclopropa (6,7)pregna-4,6-diene-21-carboxylic acid, 9,11-epoxy-6,7-dihydro-17-hydroxy-3-oxo-, monopotassium salt, (6β,7β,11α,17β)-; 3′H-cyclopropa(6,7)pregna-1,4,6-triene-21-carboxylic acid, 9,11-epoxy-6,7-dihydro-17-hydroxy-3-oxo-, γ-lactone, (6β,7β,11α,17β)-; pregn-4-ene-7,2,1-dicarboxylic acid, 9,11-epoxy-17-hydroxy-3-oxo-, γ-lactone, ethyl ester, (7α,11α,17β)-; pregn-4-ene-7,21-dicarboxylic acid, 9,11-epoxy-17-hydroxy-3-oxo-, γ-lactone, 1-methylethyl ester, (7α,11α,17β)-; RU-28318, and the like. One skilled in the art will appreciate that the aldosterone antagonists can be administered in the form of their pharmaceutically acceptable salts and/or stereoisomers. Suitable aldosterone antagonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In some embodiments, the aldosterone antagonist is eplerenone or spironolactone (a potassium sparing diuretic that acts like an aldosterone antagonist). In one embodiment eplerenone is administered in an amount of about 25 milligrams to about 300 milligrams as a single dose or as multiple doses per day; the spironolactone is administered in an amount of about 25 milligrams to about 150 milligrams as a single dose or as multiple doses per day.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) spironolactone. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) eplerenone. The compounds can be administered separately or in the form of a composition.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) one or more diuretics. Suitable diuretics include but are not limited to, thiazides (such as, for example, althiazide, bendroflumethiazide, benzclortriazide, benzhydrochlorothiazide, benzthiazide, buthiazide, chlorothiazide, cyclopenethiazide, cyclothiazide, epithiazide, ethiazide, hydrobenzthiazide, hydrochlorothiazide, hydroflumethiazide, methylclothiazide, methylcyclothiazide, penflutazide, polythiazide, teclothiazide, trichlormethiazide, triflumethazide, and the like); alilusem, ambuside, amiloride, aminometradine, azosemide, bemetizide, bumetanide, butazolamide, butizide, canrenone, carperitide, chloraminophenamide, chlorazanil, chlormerodrin, chlorthalidone, cicletanide, clofenamide, clopamide, clorexolone, conivaptan, daglutril, dichlorophenamide, disulfamide, ethacrynic acid, ethoxzolamide, etozolon, fenoldopam, fenquizone, furosemide, indapamide, mebutizide, mefruside, meralluride, mercaptomerin sodium, mercumallylic acid, mersalyl, methazolamide, meticane, metolazone, mozavaptan, muzolimine, N-(5-1,3,4-thiadiazol-2-yl)acetamide, nesiritide, pamabrom, paraflutizide, piretanide, protheobromine, quinethazone, scoparius, spironolactone, theobromine, ticlynafen, torsemide, torvaptan, triamterene, tripamide, ularitide, xipamide or potassium, AT 189000, AY 31906, BG 9928, BG 9791, C 2921, DTI 0017, JDL 961, KW 3902, MCC 134, SLV 306, SR 121463, WAY 140288, ZP 120, and the like. One skilled in the art will appreciate that the diuretics can be administered in the form of their pharmaceutically acceptable salts and/or stereoisomers. Suitable diuretics are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Depending on the diuretic employed, potassium may also be administered to the patient in order to optimize the fluid balance while avoiding hypokalemic alkalosis. The administration of potassium can be in the form of potassium chloride or by the daily ingestion of foods with high potassium content such as, for example, bananas or orange juice. The method of administration of these compounds is described in further detail in U.S. Pat. No. 4,868,179, the disclosure of which is incorporated by reference herein in its entirety.

In some embodiments, the diuretics are amiloride, furosemide, chlorthalidone, chlorothiazide, hydrochlorothiazide, hydroflumethiazide, or triamterene. In other embodiments the amiloride is administered as amiloride hydrochloride in an amount of about 5 milligrams to about 15 milligrams as a single dose or as multiple doses per day; the furosemide is administered in an amount of about 10 milligrams to about 600 milligrams as a single dose or as multiple doses per day; the chlorthalidone is administered in an amount of about 15 milligrams to about 150 milligrams as a single dose or as multiple doses per day; the chlorothiazide is administered in an amount of about 500 milligrams to about 2 grains as a single dose or as multiple doses per day; the hydrochlorothiazide is administered in an amount of about 12.5 milligrams to about 300 milligrams as a single dose or as multiple doses per day; the hydroflumethiazide is administered in an amount of about 25 milligrams to about 200 milligrams as a single dose or as multiple doses per day; the triamterene is administered in an amount of about 35 milligrams to about 225 milligrams as a single dose or as multiple doses per day.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (in) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) at least one hydralazine compound or a pharmaceutically acceptable salt thereof (e.g., hydralazine hydrochloride), (ii) at least one of isosorbide dinitrate and isosorbide mononitrate (e.g., isosorbide dinitrate), and (iii) a cardiac glycoside. The compounds can be administered separately or in the form of a composition. In one embodiment the cardiac glycoside is digoxin, acetyldigoxin, deslanoside, digitoxin or medigoxin. In other embodiments the digoxin is administered to achieve a steady state blood serum concentration of at least about 0.7 nanograms per ml to about 2.0 nanograms per ml.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof, wherein the patient has a −344 (T/T) polymorphism or a −344 (C/C) polymorphism in an aldosterone synthase CYP11B2 gene, and, optionally, at least one polymorphism in the endothelial nitric oxide synthase (NOS3) gene and/or at least one polymorphism in the beta 1 adrenergic receptor gene, comprising administering to the patient an effective amount of (i) a hydralazine compound (e.g., hydralazine hydrochloride), (ii) isosorbide dinitrate and/or isosorbide mononitrate (e.g., isosorbide dinitrate), (iii) an angiotensin-converting enzyme inhibitor selected from the group consisting of captopril, enalapril, lisinopril, ramipril, trandolapril and trandolaprilat and (iv) a β-adrenergic antagonist selected from the group consisting of carvedilol, metoprolol, bisoprolol and nebivolol. In another embodiment, the invention provides methods of administering (i) a hydralazine compound (e.g., hydralazine hydrochloride), (ii) isosorbide dinitrate and/or isosorbide mononitrate (e.g., isosorbide dinitrate), (iii) an angiotensin-converting enzyme inhibitor selected from the group consisting of enalapril, lisinopril, ramipril, trandolapril and trandolaprilat and (iv) an aldosterone antagonist selected from the group consisting of eplerenone and spironolactone. In another embodiment, the invention provides methods of administering (i) a hydralazine compound (e.g., hydralazine hydrochloride), (ii) isosorbide dinitrate and/or isosorbide mononitrate (e.g., isosorbide dinitrate), (iii) an angiotensin-converting enzyme inhibitor selected from the group consisting of captopril, enalapril, lisinopril, ramipril, trandolapril and trandolaprilat and (iv) an angiotensin II antagonist selected from the group consisting of losartan, candesartan, irbesartan and valsartan. In another embodiment, the invention provides methods of administering (i) a hydralazine compound (e.g., hydralazine hydrochloride), (ii) isosorbide dinitrate and/or isosorbide mononitrate (e.g., isosorbide dinitrate), (iii) a β-adrenergic antagonist selected from the group consisting of carvedilol, metoprolol, bisoprolol and nebivolol and (iv) an aldosterone antagonist selected from the group consisting of eplerenone and spironolactone. In another embodiment, the invention provides methods of administering (i) a hydralazine compound (e.g., hydralazine hydrochloride), (ii) isosorbide dinitrate and/or isosorbide mononitrate (e.g., isosorbide dinitrate), (iii) a β-adrenergic antagonist selected from the group consisting of carvedilol, metoprolol, bisoprolol and nebivolol and (iv) an angiotensin II antagonist selected from the group consisting of losartan, candesartan, irbesartan and valsartan. In another embodiment, the invention provides methods of administering (i) a hydralazine compound (e.g., hydralazine hydrochloride), (ii) isosorbide dinitrate and/or isosorbide mononitrate (e.g., isosorbide dinitrate), (iii) an angiotensin II antagonist selected from the group consisting of losartan, candesartan, irbesartan and valsartan (iv) a β-adrenergic antagonist selected from the group consisting of carvedilol, metoprolol, bisoprolol and nebivolol and (v) an aldosterone antagonist selected from the group consisting of eplerenone and spironolactone. In another embodiment, the invention provides methods of administering (i) a hydralazine compound (e.g., hydralazine hydrochloride), (ii) isosorbide dinitrate and/or isosorbide mononitrate (e.g., isosorbide dinitrate), (iii) an angiotensin-converting enzyme inhibitor selected from the group consisting of captopril, enalapril, ramipril, lisinopril, trandolapril and trandolaprilat (iv) a β-adrenergic antagonist selected from the group consisting of carvedilol, metoprolol, bisoprolol and nebivolol and (v) an angiotensin II antagonist selected from the group consisting of losartan, candesartan, irbesartan and valsartan. In another embodiment, the invention provides methods of administering (i) a hydralazine compound (e.g., hydralazine hydrochloride), (ii) isosorbide dinitrate and/or isosorbide mononitrate (e.g., isosorbide dinitrate), (iii) an angiotensin II antagonist selected from the group consisting of losartan, candesartan, irbesartan and valsartan and (iv) an aldosterone antagonist selected from the group consisting of eplerenone and spironolactone. In these embodiments the hydralazine compound, and at least one of isosorbide dinitrate and isosorbide mononitrate can be administered separately or as components of the same composition, and can be administered in the form of a composition with or simultaneously with, subsequently to, or prior to administration of at least one of the angiotensin converting enzyme inhibitor, β-adrenergic antagonist, angiotensin II antagonist, aldosterone antagonist, or combinations of two or more thereof. In one embodiment, all the compounds are administered together in the form of a single composition.

The invention provides methods for determining at least one polymorphism in the aldosterone synthase CYP11B2 gene in a patient followed by the administering to the patient (i) at least one antioxidant compound or pharmaceutically acceptable salt thereof; (ii) at least one nitric oxide enhancing compound; and (iii) optionally at least one compound selected from the group consisting of an angiotensin converting enzyme inhibitor, a β-adrenergic antagonist, an angiotensin II antagonist, an aldosterone antagonist, a cardiac glycoside and a diuretic compound or a combination of two or more thereof, for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; in a patient in need thereof. In these embodiments the methods include (i) obtaining a sample from a patient; (ii) analyzing the sample for at least one polymorphism in the aldosterone synthase CYP11B2 gene of a patient; and (iii) administering to the patient (a) at least one antioxidant compound or pharmaceutically acceptable salt thereof; (b) at least one nitric oxide enhancing compound; and (c) optionally at least one compound selected from the group consisting of an angiotensin converting enzyme inhibitor, a β-adrenergic antagonist, an angiotensin II antagonist, an aldosterone antagonist, a cardiac glycoside and a diuretic compound or a combination of two or more thereof. In one embodiment of the invention the sample obtained from the patient and used for the analysis of the polymorphism in the aldosterone synthase CYP11B2 gene of a patient is a blood sample. The methods to obtain a sample (e.g., blood sample) from the patient and to analyze at least one polymorphism in the aldosterone synthase CYP11B2 gene in a patient include any of the methods known to one skilled in the art, including but not limited to, those described herein.

When administered in vivo, the compounds and compositions of the invention, can be administered in combination with pharmaceutically acceptable carriers and in dosages described herein. The compounds and compositions of the invention can also be administered in combination with one or more additional compounds which are known to be effective for the treatment of heart failure or other diseases or disorders, such as, for example, anti-hyperlipidemic compounds, such as, for example, statins or HMG-CoA reductase inhibitors, such as, for example, atorvastatin (LIPITOR®), bervastatin, cerivastatin (BAYCOL®), dalvastatin, fluindostatin (Sandoz XU-62-320), fluvastatin, glenvastatin, lovastatin (MEVACOR®), mevastatin, pravastatin (PRAVACHOL®), rosuvastatin (CRESTRO®), simvastatin (ZOCOR®), velostatin (also known as synvinolin), VYTORIN™ (ezetimibe/simvastatin), GR-95030, SQ 33,600, BMY 22089, BMY 22,566, CI 980, and the like; gemfibrozil, cholystyramine, colestipol, niacin, nicotinic acid, bile acid sequestrants, such as, for example, cholestyramine, colesevelam, colestipol, poly(methyl-(3-trimethylaminopropyl) imino-trimethylene dihalide) and the like; probucol; fibric acid agents or fibrates, such as, for example, bezafibrate (Bezalip™), beclobrate, binifibrate, ciprofibrate, clinofibrate, clofibrate, etofibrate, fenofibrate (Lipidil™, Lipidil Micro™), gemfibrozil (Lopid™), nicofibrate, pirifibrate, ronifibrate, simfibrate, theofibrate and the like; cholesterol ester transfer protein (CETP) inhibitors, such as for example, CGS 25159, CP-529414 (torcetrapid), JTT-705, substituted N-[3-(1,1,2,2-tetrafluoroethoxy)benzyl]-N-(3-phenoxyphenyl)-trifluoro-3-amino-2-propanols, N,N-disubstituted trifluoro-3-amino-2-propanols, PD 140195 (4-phenyl-5-tridecyl-4H-1,2,4-triazole-3-thiol), SC-794, SC-795, SCH 58149, and the like. The hydralazine compound or pharmaceutically acceptable salt thereof, and the at least one of isosorbide dinitrate and isosorbide mononitrate, can be administered simultaneously with, subsequently to, or prior to administration of the anti-hyperlipidemic compound, or they can be administered in the form of a composition.

The compounds and compositions of the invention can be administered by any available and effective delivery system including, but not limited to, orally, bucally, parenterally, by inhalation, by topical application, by injection, transdermally, in dosage unit formulations containing conventional nontoxic pharmaceutically acceptable carriers, adjuvants, and vehicles, as desired. Parenteral includes subcutaneous injections, intravenous, intramuscular, intrasternal injection, or infusion techniques. In one embodiment of the invention the hydralazine compounds, isosorbide dinitrate and/or isosorbide mononitrate and/or therapeutic agent can be administered orally, parentally or by inhalation.

Solid dosage forms for oral administration can include capsules, sustained-release capsules, tablets, sustained release tablets, chewable tablets, sublingual tablets, effervescent tablets, pills, powders, granules and gels. In such solid dosage forms, the active compounds can be admixed with at least one inert diluent such as sucrose, lactose or starch. Such dosage forms can also comprise, as in normal practice, additional substances other than inert diluents, e.g., lubricating agents such as magnesium stearate. In the case of capsules, tablets, effervescent tablets, and pills, the dosage forms can also comprise buffering agents. Soft gelatin capsules can be prepared to contain a mixture of the active compounds or compositions of the invention and vegetable oil. Hard gelatin capsules can contain granules of the active compound in combination with a solid, pulverulent carrier such as lactose, saccharose, sorbitol, mannitol, potato starch, corn starch, amylopectin, cellulose derivatives of gelatin. Tablets and pills can be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

Injectable preparations, for example, sterile injectable aqueous or oleaginous suspensions can be formulated according to the known art using suitable dispersing agents, wetting agents and/or suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. Sterile fixed oils are also conventionally used as a solvent or suspending medium. Parenteral formulations containing compounds of the invention are disclosed in U.S. Pat. Nos. 5,530,006, 5,516,770 and 5,626,588, the disclosures of each of which are incorporated by reference herein in their entirety.

Inhaled formulations can be administered, for example, as pressurized aerosols and/or nebulized formulations to the patient's lungs. Such formulations may contain a variety of known aerosol propellants useful for endopulmonary and/or intranasal inhalation administration. In addition, water may be present, with or without any of a variety of cosolvents, surfactants, stabilizers (such as, for example, antioxidants, chelating agents, inert gases, buffers and the like). The formulation may also be aerosolized by atomizing which can produce aerosols and/or dry powder particles between 1 and 5 microns for the efficacious delivery of the inhaled formulation.

Transdermal compound administration, which is known to one skilled in the art, involves the delivery of pharmaceutical compounds via percutaneous passage of the compound into the systemic circulation of the patient. Topical administration can also involve the use of transdermal administration such as transdermal patches or iontophoresis devices. Other components can be incorporated into the transdermal patches as well. For example, compositions and/or transdermal patches can be formulated with one or more preservatives or bacteriostatic agents including, but not limited to, methyl hydroxybenzoate, propyl hydroxybenzoate, chlorocresol, benzalkonium chloride, and the like. Dosage forms for topical administration of the compounds and compositions can include creams, sprays, lotions, gels, ointments, eye drops, nose drops, ear drops, and the like. In such dosage forms, the compositions of the invention can be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions can contain polyethylene glycol 400. They can be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, can be impregnated with the compositions in solution, lotion, cream, ointment or other such form can also be used for topical application. The compositions can also be applied topically using a transdermal system, such as one of an acrylic-based polymer adhesive with a resinous crosslinking agent impregnated with the composition and laminated to an impermeable backing.

The compositions can also be applied topically using a transdermal system, such as one of an acrylic-based polymer adhesive with a resinous crosslinking agent impregnated with the composition and laminated to an impermeable backing. In a particular embodiment, the compositions of the invention are administered as a transdermal patch, more particularly as a sustained-release transdermal patch. The transdermal patches of the invention can include any conventional form such as, for example, adhesive matrix, polymeric matrix, reservoir patch, matrix or monolithic-type laminated structure, and are generally comprised of one or more backing layers, adhesives, penetration enhancers, an optional rate controlling membrane and a release liner which is removed to expose the adhesives prior to application. Polymeric matrix patches also comprise a polymeric-matrix forming material. Suitable transdermal patches are described in more detail in, for example, U.S. Pat. Nos. 5,262,165, 5,948,433, 6,010,715 and 6,071,531, the disclosure of each of which are incorporated herein in their entirety.

The compositions of this invention can further include conventional excipients, i.e., pharmaceutically acceptable organic or inorganic carrier substances suitable for parenteral application which do not deleteriously react with the active compounds. Suitable pharmaceutically acceptable carriers include, for example, water, salt solutions, alcohol, vegetable oils, polyethylene glycols, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. For parenteral application, particularly suitable vehicles consist of solutions, such as, oily or aqueous solutions, as well as suspensions, emulsions, or implants. Aqueous suspensions may contain substances which increase the viscosity of the suspension and include, for example, sodium carboxymethyl cellulose, sorbitol and/or dextran. Optionally, the suspension may also contain stabilizers.

Solvents useful in the practice of this invention include pharmaceutically acceptable, water-miscible, non-aqueous solvents. In the context of this invention, these solvents should be taken to include solvents that are generally acceptable for pharmaceutical use, substantially water-miscible, and substantially non-aqueous. The pharmaceutically-acceptable, water-miscible, non-aqueous solvents usable in the practice of this invention include, but are not limited to, N-methylpyrrolidone (NMP); propylene glycol; ethyl acetate; dimethyl sulfoxide; dimethyl acetamide; benzyl alcohol; 2-pyrrolidone; benzyl benzoate; C₂₋₆ alkanols; 2-ethoxyethanol; alkyl esters such as, 2-ethoxyethyl acetate, methyl acetate, ethyl acetate, ethylene glycol diethyl ether, or ethylene glycol dimethyl ether; (S)-(−)-ethyl lactate; acetone; glycerol; alkyl ketones such as, methylethyl ketone or dimethyl sulfone; tetrahydrofuran; cyclic alkyl amides such as, caprolactam; decylmethylsulfoxide; oleic acid; aromatic amines such as, N,N-diethyl-m-toluamide; or 1-dodecylazacycloheptan-2-one.

The pharmaceutically-acceptable, water-miscible, non-aqueous solvents include N-methylpyrrolidone (NMP), propylene glycol, ethyl acetate, dimethyl sulfoxide, dimethyl acetamide, benzyl alcohol, 2-pyrrolidone, or benzyl benzoate. Ethanol may also be used as a pharmaceutically-acceptable, water-miscible, non-aqueous solvent according to the invention, despite its negative impact on stability. Additionally, triacetin may also be used as a pharmaceutically-acceptable, water-miscible, non-aqueous solvent, as well as functioning as a solubilizer in certain circumstances. NMP may be available as PHARMASOLVE® from International Specialty Products (Wayne, N.J.), Benzyl alcohol may be available from J. T. Baker, Inc. Ethanol may be available from Spectrum, Inc. Triacetin may be available from Mallinckrodt, Inc.

The compositions of this invention can further include solubilizers. Solubilization is a phenomenon that enables the formation of a solution. It is related to the presence of amphiphiles, that is, those molecules that have the dual properties of being both polar and non-polar in the solution that have the ability to increase the solubility of materials that are normally insoluble or only slightly soluble, in the dispersion medium. Solubilizers often have surfactant properties. Their function may be to enhance the solubility of a solute in a solution, rather than acting as a solvent, although in exceptional circumstances, a single compound may have both solubilizing and solvent characteristics. Solubilizers useful in the practice of this invention include, but are not limited to, triacetin, polyethylene glycols (such as, for example, PEG 300, PEG 400, or their blend with 3350, and the like), polysorbates (such as, for example, Polysorbate 20, Polysorbate 40, Polysorbate 60, Polysorbate 65, Polysorbate 80, and the like), poloxamers (such as, for example, Poloxamer 124, Poloxamer 188, Poloxamer 237, Poloxamer 338, Poloxamer 407, and the like), polyoxyethylene ethers (such as, for example, Polyoxyl 2 cetyl ether, Polyoxyl 10 cetyl ether, and Polyoxyl 20 cetyl ether, Polyoxyl 4 lauryl ether, Polyoxyl 23 lauryl ether, Polyoxyl 2 oleyl ether, Polyoxyl 10 oleyl ether, Polyoxyl 20 oleyl ether, Polyoxyl 2 stearyl ether, Polyoxyl 10 stearyl ether, Polyoxyl 20 stearyl ether, Polyoxyl 100 stearyl ether, and the like), polyoxylstearates (such as, for example, Polyoxyl 30 stearate, Polyoxyl 40 stearate, Polyoxyl 50 stearate, Polyoxyl 100 stearate, and the like), polyethoxylated stearates (such as, for example, polyethoxylated 12-hydroxy stearate, and the like), and Tributyrin.

Other materials that may be added to the compositions of the invention include cyclodextrins, and cyclodextrin analogs and derivatives, and other soluble excipients that could enhance the stability of the inventive composition, maintain the product in solution, or prevent side effects associated with the administration of the inventive composition. Cyclodextrins may be available as ENCAPSIN® from Janssen Pharmaceuticals.

The composition, if desired, can also contain minor amounts of wetting agents, emulsifying agents and/or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, and the like.

Various delivery systems are known and can be used to administer the compounds or compositions of the invention, including, for example, encapsulation in liposomes, microbubbles, emulsions, microparticles, microcapsules and the like. The required dosage can be administered as a single unit or in a sustained release form.

The bioavailability of the compositions can be enhanced by micronization of the formulations using conventional techniques such as grinding, milling, spray drying and the like in the presence of suitable excipients or agents such as phospholipids or surfactants.

Sustained release dosage forms of the invention may comprise microparticles and/or nanoparticles having a therapeutic agent dispersed therein or may comprise the therapeutic agent in pure, preferably crystalline, solid form. For sustained release administration, microparticle dosage forms comprising pure, preferably crystalline, therapeutic agents are administered. The therapeutic dosage forms of this aspect of the invention may be of any configuration suitable for sustained release.

Nanoparticle sustained release therapeutic dosage forms can be biodegradable and, optionally, bind, to the vascular smooth muscle cells and enter those cells, primarily by endocytosis. The biodegradation of the nanoparticles occurs over time (e.g., 30 to 120 days; or 10 to 21 days) in prelysosomic vesicles and lysosomes. Larger microparticle therapeutic dosage forms of the invention release the therapeutic agents for subsequent target cell uptake with only a few of the smaller microparticles entering the cell by phagocytosis. A practitioner in the art will appreciate that the precise mechanism by which a target cell assimilates and metabolizes a dosage form of the invention depends on the morphology, physiology and metabolic processes of those cells. The size of the particle sustained release therapeutic dosage forms is also important with respect to the mode of cellular assimilation. For example, the smaller nanoparticles can flow with the interstitial fluid between cells and penetrate the infused tissue. The larger microparticles tend to be more easily trapped interstitially in the infused primary tissue, and thus are useful to deliver anti-proliferative therapeutic agents.

Particular sustained release dosage forms of the invention comprise biodegradable microparticles or nanoparticles. More particularly, biodegradable microparticles or nanoparticles are formed of a polymer containing matrix that biodegrades by random, nonenzymatic, hydrolytic scissioning to release therapeutic agent, thereby forming pores within the particulate structure.

In a particular embodiment, the compositions of the invention are administered by inhalation. For example, the inhaled formulations can comprise a therapeutically effective amount of at least one hydralazine compound or pharmaceutically acceptable salt thereof, isosorbide dinitrate and/or isosorbide mononitrate, and, optionally at least one therapeutic agent

The compounds and compositions of the invention can be formulated as pharmaceutically acceptable salt forms. Pharmaceutically acceptable salts include, for example, alkali metal salts and addition salts of free acids or free bases. The nature of the salt is not critical, provided that it is pharmaceutically acceptable. Suitable pharmaceutically-acceptable acid addition salts may be prepared from an inorganic acid or from an organic acid. Examples of such inorganic acids include, but are not limited to, hydrochloric, hydrobromic, hydroiodic, nitric, carbonic, sulfuric and phosphoric acid and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, β-hydroxybutyric, cyclohexylaminosulfonic, galactaric and galacturonic acid and the like. Suitable pharmaceutically-acceptable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound.

While individual needs may vary, determination of optimal ranges for effective amounts of the compounds and/or compositions is within the skill of the art and can be determined by standard clinical techniques, including reference to Goodman and Gilman, supra; The Physician's Desk Reference, Medical Economics Company, Inc., Oradell, N.J., 199-5; and Drug Facts and Comparisons, Inc., St. Louis, Mo., 1993. Generally, the dosage required to provide an effective amount of the compounds and compositions, which can be adjusted by one of ordinary skill in the art, will vary depending on the age, health, physical condition, sex, diet, weight, extent of the dysfunction of the recipient, frequency of treatment and the nature and scope of the dysfunction or disease, medical condition of the patient, the route of administration, pharmacological considerations such as, the activity, efficacy, pharmacokinetic and toxicology profiles of the particular compound used, whether a drug delivery system is used, and whether the compound is administered as part of a drug combination.

### EXAMPLES

### Study Population

Three hundred fifty four subjects in the African America Heart Failure Trial (A-HeFT) were enrolled in GRAHF, the Genetic Risk Assessment in Heart Failure. Inclusion criteria for A-HeFT include self designation as African Americans, heart failure due to systolic dysfunction and standard background therapy for heart failure including angiotensin converting enzyme or angiotensin receptor antagonist, and beta blockers. Subjects were randomized to either a combination of isosorbide dinitrate and hydralazine hydrochloride or placebo in addition to standard therapy. For comparisons of allele frequencies by race, the white heart cohort from GRACE (Genetic Risk Assessment of Cardiac Events), a single center investigation based at the heart failure clinic at the University of Pittsburgh, was utilized. The effect of isosorbide dinitrate and hydralazine hydrochloride on reducing mortality associate with congestive heart failure is described in U.S. Pat. Nos. 6,465,463, and 6,784,177; U.S. application Ser. Nos. 11/182,887 and 11/182,886; BiDil package insert, Final Draft 23 June (2005); BiDil NDA 20-727, FDA Advisory Committee Briefing Document, 16 June (2005), Taylor et al, New. Engl. J. Med., 351: 2049-2057 (2004), the disclosures of each of which are incorporated by reference herein in their entirety.

### Genotyping

Subjects were enrolled in GRAHF at the A-HeFT six month visit. DNA was isolated from peripheral blood by leukocyte centrifugation and cell lysis (PureGene, Gentra Systems, Minn). The aldosterone synthase (CYP11B2) promoter −344 T/C polymorphism was assessed using a TaqMan SNP Genotyping Assay with tagged primers (ABI), and products were read using the Applied Biosystems 7000 (Applied Biosystems, Foster City, Calif.).

### Outcomes Analysis

Subjects were followed to an endpoint of death or heart failure hospitalization. Quality of Life Assessment was performed by the Minnesota Living with Heart Failure Questionnaire at baseline and at the six month visit. Left ventricular function was assessed transthoracic echocardiography at baseline and six months. The primary endpoint for A-HeFT was a composite weighted score with three components: mortality, heart failure hospitalization and change in quality of life. Left ventricular remodeling was investigated in a subset of A-HeFT subjects by transthoracic echocardiography at baseline and at six months. Event free survival was compared by genotype class by Kaplan-Meier log rank analysis, using a linear model that predicts an intermediate phenotype for heterozygotes. Continuous variables such as composite scores were compared by genotype class by linear ANOVA. For the interaction of aldosterone genotype and the impact of therapy, outcomes analyzed by genotype were compared first overall and then separately by treatment subset, fixed combination of isosorbide dinitrate and hydralazine versus placebo.

### Results

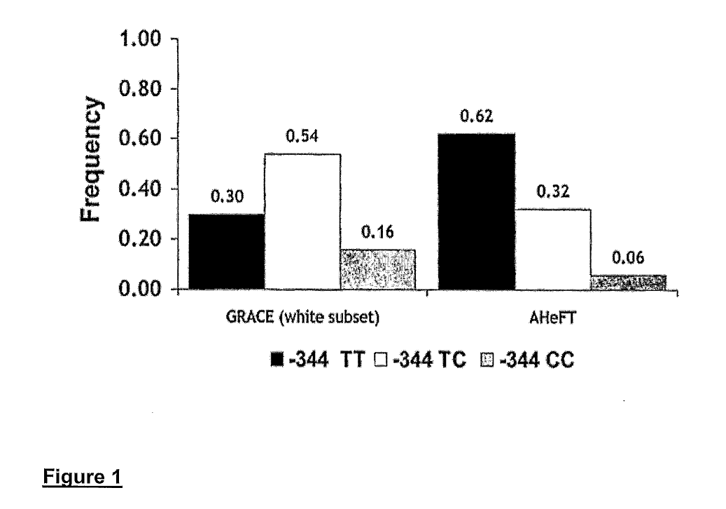

The GRAHF population was 60% male, 25% ischemic, 98% NYHA class III, with a mean age of 57. Over the course of follow up there were 60 (17%) heart failure hospitalizations and 12 deaths (3.4%). In terms of the CYP11B2-344 C/T promoter polymorphism 218 subjects (62%) were TT, 114 CT (32%), and 22 patients were CC (6%). Comparisons of etiology, medical therapy, blood pressure, and functional class were not significantly different among the three cohorts (Table 1). The allele frequencies differed markedly by race, as the T allele was much more prevalent in the black cohort in A-HEFT when compared to the white cohort from GRACE (FIG. 1, p<0.001).

TABLE 1
Patient Characteristics by −344 Genotype*

| | CC | TC | TT | All Patients |
|---|---|---|---|---|
| | (N = 22) | (N = 114) | (N = 218) | (N = 354) |
| Age (years) | 56.26 ± 12.2 | 57.9 ± 11.8 | 57.2 ± 13.5 | 57.4 ± 12.8 |
| Female (%) | 31.8 | 34.2 | 44.5 | 40.4 |
| NYHA Class | 95.5/4.5 | 96.5/3.5 | 96.8/3.2 | 96.6/3.4 |
| (%/III/IV) | | | | |
| Ischemic (%) | 22.7. | 26.3 | 25.2 | 25.4 |
| LVEE core | 0.31 ± 0.07 | 0.35 ± 0.08 | 0.35 ± 0.09 | 0.35 ± 0.09 |
| entry | | | | |
| (n = 270) | | | | |
| BP systolic | 125 ± 20 | 128 ± 17 | 127 ± 17 | 127 ± 17 |
| BP diastolic | 79 ± 14 | 78 ± 11 | 76 ± 10 | 77 ± 10 |
| Therapy | | | | |
| ACE | 72.7 | 74.6 | 77.1 | 76.0 |
| Inhibitor | | | | |
| (%) | | | | |
| Aldosterone | | | | |
| receptor | 45.5 | 33.3 | 36.5 | 36.1 |
| antagonist | | | | |
| Beta Blocker | 77.3 | 86.8.0 | 82.6 | 83.6 |
| (%) | | | | |

*No significant differences in characteristics by CYP11B2 genotype

### Event Free Survival

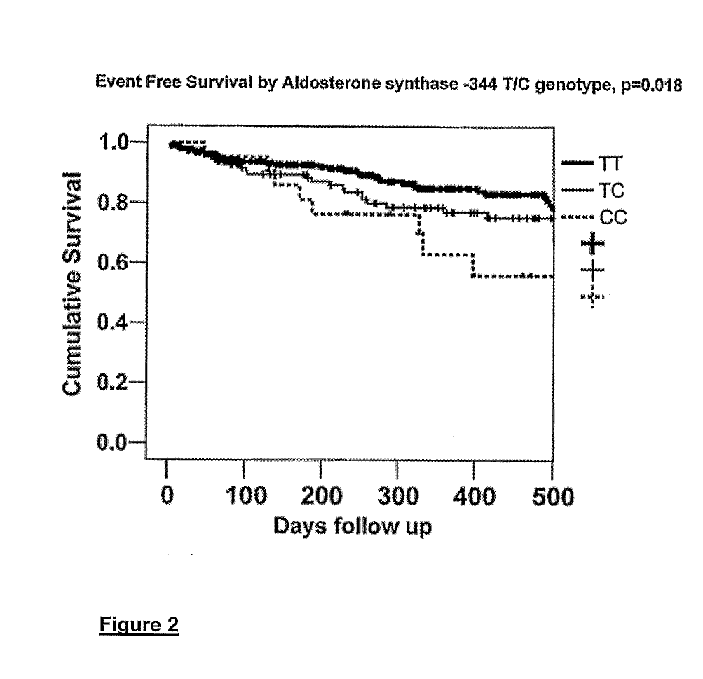

The event-free survival overall of subjects in GRAHF at 90, 180 and 360 days was 94%, 91% and 81% respectively. The C-allele was associated with significantly poorer hospitalization-free survival (FIG. 2, p=0.018) with the best survival among TT subjects (% event free survival at 90/180/360 days=94/93/85) intermediate for heterozygotes (% event free=93/90/77), and the poorest for CC homozygotes (% event free=96/81/63). Mortality was significantly greater in subjects with the C allele (% deaths TT/TC/CC=1.8%, 3.5%, 18.2%; p=0.001).

### Aldosterone Synthase Genotype, Outcomes and Isosorbide Dinitrate and Hydralazine (ISDN-HYD)

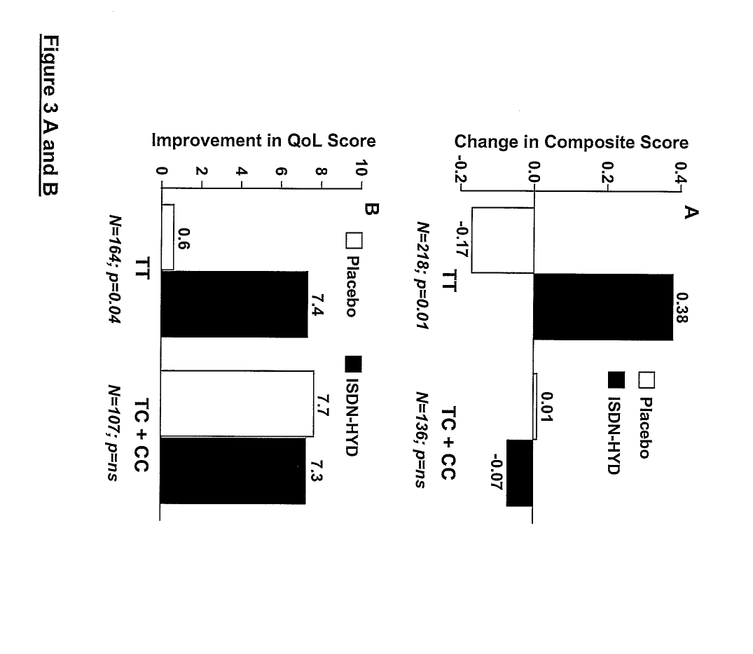

In GRAHF, treatment with isosorbide dinitrate and hydralazine hydrochloride was associated with a trend towards improved composite score (placebo=−0.09±1.7, ISDN-HYD=0.22±1.8, p=0.08). When analyzed in genotype subset, ISDN-HYD markedly improved the composite score among TT homozygotes (placebo=−0.17±1.7, ISDN-HYD=0.38±1.4, p=0.01, FIG. 3A), but had no impact among subjects with the −344C allele (placebo 0.01±1.8; ISDN-HYD=0.07±2.0, FIG. 3A). Change in Minnesota Living with Heart Failure Questionnaire (MLHFQ) Quality of Life score also suggested marked improvement in TT subjects, but not among those with the C allele (Change in MLHFQ score at 6 month from baseline=TT subset: placebo=0.6±22.6; ISDN-HYD=7.4±17.0, p=0.038; CC+TC subset: placebo=7.7±19.0; ISDN-HYD=7.3±20.4, p=ns, FIG. 3B; lower scores represents better QoL).

### Aldosterone Genotype and Left Ventricular Remodeling

Baseline ejection fraction did not differ at baseline, however there was a trend toward lower left ventricular ejection fraction (LVEF) at 6 months for subjects with the −344C allele (LVEF % for genotype subsets: TT/TC/CC=38/37/33, p=0.10, Table 2). Aldosterone antagonists did not limit the impact of the C allele on six month LVEF as in fact the impact was more pronounced for subjects on antagonists for both LVEF (−344C allele linked to lower LVEF: TT/TC/CC=39/36/32, p=0.03), and change EF from baseline to six months (□LVEF: TT/TC/CC=5.6/2.21-0.8, p=0.02). In contrast to the impact of aldosterone antagonists, a combination of isosorbide dinitrate and hydralazine hydrochloride, appeared to eliminate the impact of the C allele on remodeling, as the impact on LVEF was evident among subjects treated with placebo (LVEF: TT/TC/CC=37/36/32, p=0.05) but not for subjects on ISDN-HYD (LVEF: TT/TC/CC=38/38/40, p=0.79). Consistent with this effect of on left ventricular remodeling, subjects on placebo with the C allele had a greater left ventricular end-diastolic diameter (LVDD) at six months (LVDD (cm) TT/TC/CC=6.0/6.3/6.8, p=0.01, Table 2).

TABLE 2
Left Ventricular Ejection Fraction (LVEF) and Left Ventricular Diastolic
Diameter (LVDD) at 6 Months by −344 Genotype and Treatment

| | n = | TT | TC | CC | p-value* |
|---|---|---|---|---|---|
| LVEF | 262 | 38 ± 9 | 37 ± 9 | 33 ± 8 | ns (0.10) |
| LVDD (cm) | 268 | 6.1 ± 1.3 | 6.2 ± 1.3 | 6.5 ± 1.3 | ns (0.22) |
| LVEF | 99 | 39 ± 10 | 36 ± 9 | 32 ± 6 | 0.03 |
| on spironolactone | | | | | |
| LVDD (cm) | 101 | 6.2 ± 1.4 | 6.3 ± 1.5 | 6.5 ± 0.8 | ns (0.51) |
| on spironolactone | | | | | |
| LVEF | 136 | 37 ± 10 | 36 ± 10 | 32 ± 7 | 0.05 |
| on placebo | | | | | |
| LVDD (cm) | 141 | 6.0 ± 1.3 | 6.3 ± 1.2 | 6.8 ± 0.9 | 0.01 |
| on placebo | | | | | |
| LVEF | 126 | 38 ± 9 | 38 ± 8 | 40 ± 6 | ns (0.79) |
| on ISDN-HYD | | | | | |
| LVDD (cm) | 131 | 6.2 ± 1.4 | 6.0 ± 1.4 | 5.6 ± 1.9 | ns (0.34) |
| on ISDN-HYD | | | | | |

*comparisons of means by linear ANOVA

### Discussion

In the GRAHF study, the CYP11B2 promoter −344C allele linked to higher expression of aldosterone synthase was associated with increase risk of death and hospitalization for African American subjects heart failure. Analysis by treatment subset suggests the C allele also worsens LV remodeling. Of interest, the impact of the NO donor strategy ISDN-HYD was greatest in subjects with the TT genotype, a genotype predominant in African Americans and previously linked to low-renin hypertension. The results of this investigation suggest that genetic variation in aldosterone production plays an important role in left ventricular remodeling and disease progression in African American with heart failure, a population underrepresented in previous genetics outcomes investigations.

While the −344C allele in vitro has increased binding of SF-1, the impact on transcriptional activity and aldosterone levels in vivo remains controversial. In clinical studies in essential hypertension, the C allele was associated with higher circulating levels of aldosterone in a gene ordered fashion with the highest levels in the CC genotype subset, intermediate in heterozygotes and lowest in TT homozygotes. However, the linkage of the −344 genotype with aldosterone levels has been inconsistent as several reports actually associate the −344T allele with higher levels. An analysis from the Framingham study suggests the variance in aldosterone levels in populations is primarily due to non-genetic factors. The impact of the CYP11B2 genotype on aldosterone levels may be dependent on a subjects overall level of neurohormonal activation. While these previous studies have been in normal subjects or in those with hypertension, little data exists on the interaction of the −344 T/C polymorphism with aldosterone levels in heart failure cohorts.

As shown herein, the −344 T allele is consistently more prevalent in black cohorts. The heart failure phenotype differs in African American and white cohorts, with a much greater prevalence of hypertensive cardiomyopathy. Low renin hypertension, in which the aldoterone/renin ratio is elevated, is particularly more prevalent in African Americans and has been linked to the T allele. In a hypertension study comparing the aldosterone antagonist eplerenone to angiotensin II antagonists (ARBs), eplerenone was more effective than angiotensin II antagonists in black cohorts. Whether aldosterone antagonists are more effective in African Americans as heart failure therapy will require further investigation

Stimulation of the myocardium by aldosterone induces left ventricular remodeling, hypertrophy and fibrosis. In a Finnish cohort free of cardiac disease, the −344 C allele was associated with increased LV size and mass. In a study of 995 members of 229 families, the CYP11B2 haplotype was linked to LV cavity size and wall thickness. In the current GRAHF cohort, the C allele was associated with a trend towards lower LVEF at 6 months. This was particularly significant for the subset on placebo and was not evident for subjects randomized to a combination of isosorbide dinitrate and hydralazine hydrochloride. Of note, the impact of the C allele was more pronounced for subjects on aldosterone receptor antagonists. However aldosterone receptor antagonists were not randomized in GRAHF, and their use may represent a marker for a higher risk subjects rather than a pharmacogenetic interaction. Overall the current study is consistent with previous reports of an increase risk of remodeling with the −344 C allele, and this may be the mechanism of its adverse effect on heart failure outcomes.

Low renin hypertension is associated with endothelial dysfunction and has been linked to the −344 TT genotype. Aldosterone excess in low renin states is associated with impaired nitric oxide mediated vasodilation. Treatment with aldosterone antagonists increase NOS3 levels, which may help to restore endothelial function and contribute to their therapeutic effects in subjects with heart failure. In V-HeFT I the therapeutic impact of treatment with a combination of isosorbide dinitrate and hydralazine hydrochloride was greater in the African Americans cohort, and this finding was confirmed by the marked benefits in heart failure survival with treatment in A-HeFT. In GRAHF, the impact of therapy with IDN-HYD was primarily in the −344 TT genotype predominant in African Americans and linked to low renin hypertension. Whether nitric oxide donor strategies are more effective in low-renin states, and the interaction of aldosterone with nitric oxide and oxidative stress in heart failure remains to be determined.

The study described herein has a number of limitations. Circulating mediators were not evaluated as part of GRAHF so the impact of the −344 T/C polymorphism on aldosterone level was not investigated. Treatment with aldoterone receptor antagonists was not randomized and was utilized in a minority of subjects, so the impact of CYP11B2 genotype on treatment designed to block aldosterone could not be evaluated. Finally, the mortality rate for subjects in GRAHF (3.4%) was lower than in the A-HeFT trial itself, and this limited the ability in this smaller cohort to evaluate the impact of genotype on survival as a single endpoint.

The current investigation demonstrates that the −344 T/C promoter polymorphism of CYP11B2 influences clinical outcomes in an African American cohort with heart failure, and provides evidence for the importance of aldosterone in heart failure progression. The heart failure phenotype for African Americans differs from whites and the allele frequencies of this functional polymorphism differ markedly in black and white cohorts. These results from GRAHF suggest genetic variation in aldosterone production may contribute to these phenotypic differences. In determining optimal heart failure treatment for an individual, race is likely a surrogate marker for differences in genetic background. Future investigations may prove that genomic analysis is a more accurate tool for tailoring of heart failure therapies than racial designation.

The disclosure of each patent, patent application and publication cited or described in the present specification is hereby incorporated by reference herein in its entirety.

Although the invention has been set forth in detail, one skilled in the alt will appreciate that numerous changes and modifications can be made to the invention, and that such changes and modifications can be made without departing from the spirit and scope of the invention.

1. A method for reducing mortality associated with heart failure; improving oxygen consumption; treating heart failure; treating hypertension; improving the quality of life in a heart failure patient; inhibiting left ventricular remodeling; reducing a hospitalization related to heart failure; improving exercise tolerance; increasing left ventricular ejection fraction; decreasing levels of B-type natriuretic protein; treating a renovascular disease; treating an end-stage renal disease; reducing cardiomegaly; treating a disease resulting from oxidative stress; treating an endothelial dysfunction; treating a disease caused by endothelial dysfunctions; or treating a cardiovascular disease in a patient in need thereof, comprising administering to the patient (i) at least one antioxidant compound or a pharmaceutically acceptable salt thereof; and (ii) at least one nitric oxide enhancing compound, wherein the patient has a −344 (T/T) polymorphism or a −344 (C/C) polymorphism in an aldosterone synthase CYP11B2 gene.

2. The method of claim 1, wherein the patient has at least one polymorphism in the endothelial nitric oxide synthase (NOS3) gene and/or at least one polymorphism in the beta 1 adrenergic receptor gene.

3. The method of claim 2, wherein the at least one polymorphism in the endothelial nitric oxide synthase (NOS3) gene is an Asp298Glu polymorphism in exon 7 of the endothelial nitric oxide synthase gene, a T-786C polymorphism in the promoter region of the endothelial nitric oxide synthase-gene or a 27 base-pair tandem repeat intron 4 polymorphism of the endothelial nitric oxide synthase gene.

4. The method of claim 3, wherein the Asp298Glu polymorphism in exon 7 of the endothelial nitric oxide synthase gene is a Glu298Glu variant; the T-786C polymorphism in the promoter region of the endothelial nitric oxide synthase gene is a T-786C variant or a T-786T variant; and the intron 4 polymorphism in the endothelial nitric oxide synthase gene is an intron 4a/4b variant or an intron 4b/4b valiant.

5. The method of claim 2, wherein the at least one polymorphism in the beta 1 adrenergic receptor gene is an Arg389Arg polymorphism and/or a Gly389Gly polymorphism in the beta 1 adrenergic receptor gene.

6. The method of claim 1, wherein the least one nitric oxide enhancing compound is isosorbide dinitrate or isosorbide mononitrate.

7. The method of claim 1, wherein the antioxidant is a hydralazine compound or a pharmaceutically acceptable salt thereof.

8. The method of claim 7, wherein the hydralazine compound is hydralazine hydrochloride.

9. The method of claim 1, comprising administering an effective amount of hydralazine hydrochloride and isosorbide dinitrate; wherein the hydralazine hydrochloride and the isosorbide dinitrate are administered separately or as components of the same composition.

10. The method of claim 9, wherein the hydralazine hydrochloride and the isosorbide dinitrate are administered in a sustained release form.

11. The method of claim 9, comprising orally administering to the patient hydralazine hydrochloride in an amount of about 30 milligrams to about 400 milligrams and isosorbide dinitrate in an amount of about 10 milligrams to about 200 milligrams.

12. The method of claim 9, comprising administering (i) 37.5 mg hydralazine hydrochloride and 20 milligrams isosorbide dinitrate or (ii) 75 mg hydralazine hydrochloride and 40 milligrams isosorbide dinitrate.

13. The method of claim 9, comprising administering (i) hydralazine hydrochloride in an amount of about 225 milligrams per day and isosorbide dinitrate in an amount of about 120 milligrams per day; or (ii) hydralazine hydrochloride in an amount of about 112.5 milligrams once or twice per day and isosorbide dinitrate in an amount of about 60 milligrams once or twice per day.

14. The method of claim 9, comprising administering (i) hydralazine hydrochloride in an amount of about 75 milligrams once, twice or three times per day and isosorbide dinitrate in an amount of about 40 milligrams once, twice or three times per day, or (ii) hydralazine hydrochloride in an amount of about 37.5 milligrams once, twice or three times per day and isosorbide dinitrate in an amount of about 20 milligrams once, twice or three times per day; wherein the hydralazine hydrochloride and the isosorbide dinitrate are administered separately or as components of the same composition.

15. The method of claim 1, further comprising administering at least one aldosterone antagonist.

16. The method of claim 15, wherein the aldosterone antagonist is eplerenone or spironolactone

17. The method of claim 1, further comprising administering at least one compound selected from the group consisting of an angiotensin converting enzyme inhibitor, a β-adrenergic antagonist, an angiotensin II antagonist, an aldosterone antagonist, a cardiac glucoside, a diuretic compound or a combination of two or more thereof.

18. The method of claim 1, further comprising administering captopril, enalapril, lisinopril, ramipril, metoprolol, carvidilol, nebivolol, spironolactone or eplerenone.

19. The method of claim 1, wherein the patient is categorized as New York Heart Association heart failure functional classification I, II, III or IV.

20. The method of claim 1, wherein the patient is a black patient.
